SEQUENCE LISTING (21 sequences)

SEQ ID NO: 1 - DNA, 820 bp, homo sapiens
gccctggcaaggttgtgggggacatcttgagctgaagcagggttttgagccactgctgctgctgccattgtcaccatggtctcagctctgcggggagcacccctgatcagggtgcactcaagccctgtttcttctccttctgtgagtggaccacggaggctggtgagctgcctgtcatcccaaagctcagctctgagccagagtggtggtggctccacctctgccgccggcatagaagccaggagcagggctctcagaaggcggtggtgcccagctgggatcatgttgttggccctggtctgtctgctcagctgcctgctaccctccagtgaggccaagctctacggtcgttgtgaactggccagagtgctacatgacttcgggctggacggataccggggatacagcctggctgactgggtctgccttgcttatttcacaagcggtttcaacgcagctgctttggactacgaggctgatgggagcaccgacaacgggatcttccagatcaacagccggaggtggtgcagcaacctcaccccgaacgtccccaacgtgtgccggatgtactgctcagatttgttgaatcctaatctcaaggataccgttatctgtgccatgaagataacccaagagcctcagggtctgggttactgggaggcctggaggcatcactgccagggaaaagacctcactgaatgggtggatggctgtgacttctaggatggacggaaccatgcacagcaggctgggaaatgtggtttggttcctgacctaggcttgggaagacaagccagcgaataaaggatggttgaacgtt

SEQ ID NO: 2 - protein, 215 aa, homo sapiens
MVSALRGAPLIRVHSSPVSSPSVSGPRRLVSCLSSQSSALSQSGGGSTSAAGIEARSRALRRRWCPAGIMLLALVCLLSCLLPSSEAKLYGRCELARVLHDFGLDGYRGYSLADWVCLAYFTSGFNAAALDYEADGSTDNGIFQINSRRWCSNLTPNVPNVCRMYCSDLLNPNLKDTVICAMKITQEPQGLGYWEAWRHHCQGKDLTEWVDGCDF

SEQ ID NO: 3 - DNA, 588 bp, homo sapiens
ctgggagggcttacaggtgccataatgaaggcctggggcactgtggtagtgaccttggccacgctgatggttgtcactgtggatgccaagatctatgaacgctgcgagctggcggcaagactggagagagcagggctgaacggctacaagggctacggcgttggagactggctgtgcatggctcattatgagagtggctttgacaccgccttcgtggaccacaatcctgatggcagcagtgaatatggcattttccaactgaattctgcctggtggtgtgacaatggcattacacccaccaagaacctctgccacatggattgtcatgacctgctcaatcgccatattctggatgacatcaggtgtgccaagcagattgtgtcctcacagaatgggctttctgcctggacttcttggaggctacactgttctggccatgatttatctgaatggctcaaggggtgtgatatgcatgtgaaaattgatccaaaaattcatccatgactcagattcgaagagacagattttatcttcctttcatttctttctcttgtgcatttaataaaggatggtatctataaacaatgc

SEQ ID NO: 4 - protein, 159 aa, homo sapiens
MKAWGTVVVTLATLMVVTVDAKIYERCELAARLERAGLNGYKGYGVGDWLCMAHYESGFDTAFVDHNPDGSSEYGIFQLNSAWWCDNGITPTKNLCHMDCHDLLNRHILDDIRCAKQIVSSQNGLSAWTSWRLHCSGHDLSEWLKGCDMHVKIDPKIHP

SEQ ID NO: 5 - DNA, 387 bp, homo sapiens
agcctcatcagtcgctgtgacttggcccaggtgctgcagctggaggacttggatgggtttgagggttactccctgagtgactggctgtgcctggcttttgtggaaagcaagttcaacatatcaaagataaatgaaaatgcagatggaagctttgactatggcctcttccagatcaacagccactactggtgcaacgattataagagttactcggaaaacctttgccacgtagactgtcaagatctgctgaatcccaaccttcttgcaggcatccactgcgcaaaaaggattgtgtccggagcacgggggatgaacaactgggtagaatggaggttgcactgttcaggccggccactcttctactggctgacaggatgccgcctgaga

SEQ ID NO: 6 - protein, 148 aa, homo sapiens
MTKALLIYLVSSFLALNQASLISRCDLAQVLQLEDLDGFEGYSLSDWLCLAFVESKFNISKINENADGSFDYGLFQINSHYWCNDYKSYSENLCHVDCQDLLNPNLLAGIHCAKRIVSGARGMNNWVEWRLHCSGRPLFYWLTGCRLR

SEQ ID NO: 7 - DNA, 790 bp, homo sapiens
agaatcagacatggcttcaggggatgcaggacgctcccctgagctgcctgtcaccgactaggtggagcagtgtttcttccgcagactcaactgagaagtcagcctctggggcaggcaccaggaatctgccttttcagttctgtctccggcaggctttgaggatgaaggctgcgggcattctgaccctcattggctgcctggtcacaggcgccgagtccaaaatctacactcgttgcaaactggcaaaaatattctcgagggctggcctggacaattactggggcttcagccttggaaactggatctgcatggcatattatgagagcggctacaacaccacagccccgacggtcctggatgacggcagcatcgactatggcatcttccagatcaacagcttcgcgtggtgcagacgcggaaagctgaaggagaacaaccactgccatgtcgcctgctcagccttgatcactgatgacctcacagatgcaattatctgtgccaggaaaattgttaaagagacacaaggaatgaactattggcaaggctggaagaaacattgtgagggcagagacctgtccgagtggaaaaaaggctgtgaggtttcctaaactggaactggacccaggatgctttgcagcaacgccctaggatttgcagtgaatgtccaaatgcctgtgtcatcttgtcccgtttcctcccaatattccttctcaaacttggagagggaaaattaagctatacttttaagaaaataaatatttccatttaaatgtcaaaaaaaaaaaaaaaa

SEQ ID NO: 8 - protein, 194 aa, homo sapiens
MQDAPLSCLSPTRWSSVSSADSTEKSASGAGTRNLPFQFCLRQALRMKAAGILTLIGCLVTGAESKIYTRCKLAKIFSRAGLDNYWGFSLGNWICMAYYESGYNTTAPTVLDDGSIDYGIFQINSFAWCRRGKLKENNHCHVACSALITDDLTDAIICARKIVKETQGMNYWQGWKKHCEGRDLSEWKKGCEVS

SEQ ID NO: 9 - DNA, 758 bp, homo sapiens
cgctcccctgagctgcctgtcaccgactaggtggagcagtgtttcttccgcagactcaactgagaagtcagcctctggggcaggcaccaggaatctgccttttcagttctgtctccggcaggctttgaggatgaaggctgcgggcattctgaccctcattggctgcctggtcacaggcgccgagtccaaaatctacactcgttgcaaactggcaaaaatattctcgagggctggcctggacaattactggggcttcagccttggaaactggatctgcatggcgtattatgagagcggctacaacaccacagcccagacggtcctggatgacggcagcatcgactacggcatcttccagatcaacagcttcgcgtggtgcagacgcggaaagctgaaggagaacaaccactgccacgtcgcctgctcagccttggtcactgatgacctcacagatgcgattatctgtgccaagaaaattgttaaagagacacaaggaatgaactattggcaaggctggaagaaacactgtgaggggagagacctgtccgactggaaaaaaggctgtgaggtttcctaaactggaactggacccaggatgctttgcagcaacgccctaggatttgcagtgaatgtccaaatgcctgtgtcatcttgtcccgtttcctcccaatattccttctcaaacttggagagggaaaattaagctatacttttaagaaaataaatatttccatttaaatgtcaaaaaaaaaaaaaaa

SEQ ID NO: 10 - protein, 148 aa, homo sapiens
MKAAGILTLIGCLVTGAESKIYTRCKLAKIFSRAGLDNYWGFSLGNWICMAYYESGYNTTAQTVLDDGSIDYGIFQINSFAWCRRGKLKENNHCHVACSALVTDDLTDAIICAKKIVKETQGMNYWQGWKKHCEGRDLSDWKKGCEVS

SEQ ID NO: 11 - DNA, 886 bp, homo sapiens
agtggatgattaatgaagtagcatctgaaaaacccaaggaaccaaatgtaggtggagcaacttccctgcaacacacgtcgaagctgctgcagtggctgttcttcccctccccttggaaaggcaccaaaagccagcctttggaagggctggtggactgtgcccttctttcccttcagagaaccagtgtccctgtgactccacccactcatctggccaccgttgccctgacctgccaggagcctggagaagatgaaggcatccgtggttctctccctccttggctacctggtggttccaagtggtgcttacatcttggggcgttgcacagtggctaagaaactccacgatggaggcctggattattttgagggctatagccttgagaactgggtgtgcctggcctacttcgagagcaagttcaaccccatggccatctacgagaacacacgtgagggctacactggctttggcctctttcagatgcgtggcagtgactggtgtggcgaccatggcaggaaccgctgccatatgtcatgttccgctttactgaatcctaatttagagaagacaattaaatgtgccaagaccattgtaaaaggaaaagaagggatgggagcatggcccacctggtcccggtactgccagtactccgataccctggcacggtggctggatggttgcaagctgtagccgcctgcatggcccctgcagcactcaccagttgcatcttgtgaatgaaggtgcttttctgcttgctgcttcagtcaatccttttgatgatctcaccactttaagagttccagatggaaaaagacaaaagtttgcttcatccggggatgcaggatgcagaataaaccaaactagttactcaaaaaaaaaaaaaaaa

SEQ ID NO: 12 - protein, 146 aa, homo sapiens
MKASVVLSLLGYLVVPSGAYILGRCTVAKKLHDGGLDYFEGYSLENWVCLAYFESKFNPMAIYENTREGYTGFGLFQMRGSDWCGDHGRNRCHMSCSALLNPNLEKTIKCAKTIVKGKEGMGAWPTWSRYCQYSDTLARWLDGCKL

SEQ ID NO: 13 - protein, 221 aa, Mus musculus
MGICMSMYTQVLVPVDADGDHHILWSRFYERWGSCFNPCAGLVNCLPPHSSALYLCHRMEPRSRASIRQLCPPGITWLALAYLLSCLLASSKAKVFSRCELAKEMHDFGLDGYRGYNLADWVCLAYYTSGFNTNAVDHEADGSTNNGIFQISSRRWCRTLASNGPNLCRIYCTDLLNNDLKDSIVCAMKIVQEPLGLGYWEAWRHHCQGRDLSDWVDGCDF

SEQ ID NO: 14 - DNA, 36 bp, Artificial Sequence
  Primer for PCR cloning of mSLLP1
catgccatggccaaggtcttcagtcgctgtgagctg

SEQ ID NO: 15 - DNA, 33 bp, Artificial Sequence
  Primer for PCR cloning of mSLLP1
ccgctcgaggaagtcacagccatccacccagtc

SEQ ID NO: 16 - protein, 128 aa, homo sapiens
KLYGRCELARVLHDFGLDGYRGYSLADWVCLAYFTSGFNAAALDYEADGSTDNGIFQINSRRWCSNLTPNVPNVCRMYCSDLLNPNLKDTVICAMKITQEPQGLGYWEAWRHHCQGKDLTEWVDGCDF

SEQ ID NO: 17 - protein, 138 aa, homo sapiens
KIYERCELAARLERAGLNGYKGYGVGDWLCMAHYESGFDTAFVDHNPDGSSEYGIFQLNSAWWCDNGITPTKNLCHMDCHDLLNRHILDDIRCAKQIVSSQNGLSAWTSWRLHCSGHDLSEWLKGCDMHVKIDPKIHP

SEQ ID NO: 18 - protein, 129 aa, homo sapiens
SLISRCDLAQVLQLEDLDGFEGYSLSDWLCLAFVESKFNISKINENADGSFDYGLFQINSHYWCNDYKSYSENLCHVDCQDLLNPNLLAGIHCAKRIVSGARGMNNWVEWRLHCSGRPLFYWLTGCRLR

SEQ ID NO: 19 - protein, 129 aa, homo sapiens
KIYTRCKLAKIFSRAGLDNYWGFSLGNWICMAYYESGYNTTAPTVLDDGSIDYGIFQINSFAWCRRGKLKENNHCHVACSALITDDLTDAIICARKIVKETQGMNYWQGWKKHCEGRDLSEWKKGCEVS

SEQ ID NO: 20 - protein, 129 aa, homo sapiens
KIYTRCKLAKIFSRAGLDNYWGFSLGNWICMAYYESGYNTTAQTVLDDGSIDYGIFQINSFAWCRRGKLKENNHCHVACSALVTDDLTDAIICAKKIVKETQGMNYWQGWKKHCEGRDLSDWKKGCEVS

SEQ ID NO: 21 - protein, 127 aa, homo sapiens
YILGRCTVAKKLHDGGLDYFEGYSLENWVCLAYFESKFNPMAIYENTREGYTGFGLFQMRGSDWCGDHGRNRCHMSCSALLNPNLEKTIKCAKTIVKGKEGMGAWPTWSRYCQYSDTLARWLDGCKL

(19) United States
(12) Patent Application Publication
(10) Pub. No.: US 2006/0089297 A1
(43) Pub. Date: Apr. 27, 2006

(54) Sperm specific lysozyme-like proteins

(76) Inventors: John C. Herr, Charlottesville, VA (US); Maria Belen Herrero, Alexandria, VA (US); Arabinda Mandal, Charlottesville, VA (US); Laura Clayton Digilio, Crozet, VA (US)

Correspondence Address: UNIVERSITY OF VIRGINIA PATENT FOUNDATION, 250 WEST MAIN STREET, SUITE 300, CHARLOTTESVILLE, VA, 22902, US

(21) Appl. No.: 10/542,038

(22) PCT Filed: Jan. 16, 2004
(86) PCT No.: PCT/US04/01240

§ 371 Date: Jul. 13, 2005

Related U.S. Application Data
(60) Provisional application No. 60/440,585, filed on Jan. 16, 2003.

(51) Int. Cl. A61K 38/17; C07K 14/47

(52) U.S. Cl. 514/2; 530/350; 530/388.1

(57) ABSTRACT

The present invention is directed to a family of testis specific proteins (SLLPs) that share high sequence identity to lysozyme-C proteins. The application encompasses compositions comprising the SLLP proteins, antibodies specific for the SLLP polypeptides and the use of the SLLP polypeptides and antibodies directed to such peptides as contraceptive agents.

### RELATED APPLICATIONS

This application claims priority under 35 USC §119(e) to U.S. Provisional Application Ser. No. 60/440,585, filed Jan. 16, 2003, the disclosure of which is incorporated herein by reference.

### U.S. GOVERNMENT RIGHTS

This invention was made with United States Government support under Grant Nos. HID 38353, and U54 29099, awarded by National Institutes of Health. The United States Government has certain rights in the invention.

### BACKGROUND

During fertilization in mammals, capacitated spermatozoa must first penetrate the mass of cumulus cells surrounding the oocyte and then the thick extracellular matrix of the zona pellucida. Spermatozoa that reach and bind to the zona pellucida receive a signal to undergo the acrosome reaction, releasing enzymes that act to facilitate hydrolysis of a fertilization channel through the zona pellucida. Upon emergence from the fertilization channel, acrosome-reacted spermatozoa cross the perivitelline space and bind to and fuse with the oolemma. Only acrosome-reacted sperm are found in the peri-vitelline matrix and only acrosome-reacted sperm are fusogenic with the plasmalemma domain overlying the equatorial segment currently thought to mediate binding and fusion events. Thus, fertilization is completed through direct interactions between sperm and oocyte surface proteins. The gamete ligands and receptors and the molecular interactions essential to these events are the subject of much research effort.

As described in the International Application No. PCT/US01/01716, the disclosure of which is incorporated herein, applicants previously discovered two novel c-type lysozyme-like proteins (hSLLP1 & hSLLP2) present in the human acrosome. An additional 4 members of this gene family have now been isolated (hSLLP3-6). The expression of each of the SLLP family members is limited to the testes and as described herein appears to function in the binding and fusion of the sperm and oocyte membranes. Accordingly, one aspect of the present invention is directed the use of these proteins as targets for isolating contractive agents.

### SUMMARY OF VARIOUS EMBODIMENTS OF THE INVENTION

The present invention is directed to six sperm-specific lysozyme-like proteins designated SLLP1, SLLP2, SLLP3 (previously named C19, C23 and C24, respectively), SLLP4, SLLP5 and SLLP6, nucleic acid sequences encoding those proteins, and antibodies generated against said proteins. Compositions comprising the native SLLP1, SLLP2, SLLP3, SLLP4, SLLP5 and SLLP6 peptides can be used in contraceptive formulations. Furthermore, antibodies generated against SLLP1, SLLP2, SLLP3, SLLP4, SLLP5 and SLLP6 can be used as diagnostic agents or can be formulated in compositions that are used to interfere with the binding of sperm cells to oocytes.

### BRIEF DESCRIPTION OF THE DRAWINGS

FIG. 1 is a bar graph representing data obtained from the mouse sperm-egg binding experiment described in Example 3. Zona pellucidae from mature mouse eggs were removed by brief incubation in chymotrypsin followed by mechanical shearing. Capacitated mouse sperm, pre-incubated with different concentrations of anti-recmSLLP1 sera were co-incubated with zona-free mouse eggs and the number of sperm cells binding to the egg was determined.

FIG. 2 is a bar graph representing data obtained from the mouse sperm-egg fusion experiment described in Example 3. Zona pellucidae from mature mouse eggs were removed by brief incubation in chymotrypsin followed by mechanical shearing. Capacitated mouse sperm, pre-incubated with different concentrations of anti-recmSLLP1 sera were co-incubated with zona-free mouse eggs and the number of sperm cells fused with the egg membrane was determined.

FIG. 3 is a bar graph representing data obtained from an experiment studying the effect of mouse recombinant SLLP1 on mouse sperm-egg binding (see Example 3). Zona-free eggs were pre-incubated with the indicated concentrations of recmSLLP1 protein and then inseminated with capacitated mouse sperm. In all cases, the sera or the recombinant proteins were present during gamete interaction. Eggs were processed and analyzed for sperm binding. Data represent the mean±SE from three [symbol] experiments. (*) P≦0.05, (**) p≦0.01 (Student's T test). Controls: preiimune sera or no protein or recePAD (a cytoplasmic egg protein).

FIG. 4 is a bar graph representing data obtained from an experiment studying the effect of mouse recombinant SLLP1 on mouse sperm-egg fusion (see Example 3). Zona-free eggs were pre-incubated with the indicated concentrations of recmSLLP1 protein and then inseminated with capacitated mouse sperm. In all cases, the sera or the recombinant proteins were present during gamete interaction. Eggs were processed and analyzed for sperm fusion. Data represent the mean±SE from three different experiments. (*) P≦0.05, (**) P≦0.01 (Student's T test). Controls: preiimune sera or no protein or recePAD (a cytoplasmic egg protein).

FIG. 5 is a bar graph representing data obtained from an experiment studying the effect of human recombinant SLLP1 on mouse sperm-egg binding (see Example 4). Zona-free eggs were pre-incubated with the indicated concentrations of rechSLLP1 protein and then inseminated with capacitated mouse sperm. In all cases, the sera or the recombinant proteins were present during gamete interaction. Eggs were processed and analyzed for sperm binding. Data represent the mean±SE from three different experiments. (*) P≦0.05, (**) P≦0.01 (Student's T test). Controls: preiimune sera or no protein or recePAD (a cytoplasmic egg protein).

FIG. 6 is a bar graph representing data obtained from an experiment studying the effect of human recombinant SLLP1 on mouse sperm-egg fusion (see Example 4). Zona-free eggs were pre-incubated with the indicated concentrations of recmSLLP1 protein and then inseminated with capacitated mouse sperm. In all cases, the sera or the recombinant proteins were present during gamete interaction. Eggs were processed and analyzed for sperm fusion. Data represent the mean±SE from three different experiments. (*) P≦0.05, (**) P≦0.01 (Student's T test). Controls: preiimune sera or no protein or recePAD (a cytoplasmic egg protein).

### DETAILED DESCRIPTION OF EMBODIMENTS

### DEFINITIONS

In describing and claiming the invention, the following terminology will be used in accordance with the definitions set forth below.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

A polylinker is a nucleic acid sequence that comprises a series of three or more closely spaced restriction endonuclease recognitions sequences. “Operably linked” refers to a juxtaposition wherein the components are configured so as to perform their usual function. Thus, control sequences or promoters operably linked to a coding sequence are capable of effecting the expression of the coding sequence.

As used herein, “nucleic acid,” “DNA,” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

The term “peptide” encompasses a sequence of 3 or more amino acids wherein the amino acids are naturally occurring or synthetic (non-naturally occurring) amino acids. Peptide mimetics include peptides having one or more of the following modifications:

1. peptides wherein one or more of the peptidyl —C(O)NR— linkages (bonds) have been replaced by a non-peptidyl linkage such as a —CH₂-carbamate linkage (—CH₂OC(O)NR—), a phosphonate linkage, a —CH₂-sulfonamide (—CH₂—S(O)₂NR—) linkage, a urea (—NHC(O)NH—) linkage, a —CH₂-secondary amine linkage, or with an alkylated peptidyl linkage (—C(O)NR—) wherein R is C₁-C₄ alkyl;

2. peptides wherein the N-terminus is derivatized to a —NRR, group, to a —NRC(O)R group, to a —NRC(O)OR group, to a —NRS(O)₂R group, to a —NHC(O)NHR group where R and R₁ are hydrogen or C₁-C₄ alkyl with the proviso that R and R₁ are not both hydrogen;

3. peptides wherein the C terminus is derivatized to —C(O)R₂ where R₂ is selected from the group consisting of C₁-C₄ alkoxy, and —NR₃R₄ where R₃ and R₄ are independently selected from the group consisting of hydrogen and C₁-C₄ alkyl.

Naturally occurring amino acid residues in peptides are abbreviated as recommended by the IUPAC—IUB Biochemical Nomenclature Commission as follows: Phenylalanine is Phe or F; Leucine is Leu or L; Isoleucine is Ile or I; Methionine is Met or M; Norleucine is Nle; Valine is Val or V; Serine is Ser or S; Proline is Pro or P; Threonine is Thr or T; Alanine is Ala or A; Tyrosine is Tyr or Y; Histidine is His or H; Glutamine is Gln or Q; Asparagine is Asn or N; Lysine is Lys or K; Aspartic Acid is Asp or D; Glutamic Acid is Glu or E; Cysteine is Cys or C; Tryptophan is Trp or W; Arginine is Arg or R; Glycine is Gly or G, and X is any amino acid. Other naturally occurring amino acids include, by way of example, 4-hydroxyproline, 5-hydroxylysine, and the like.

Synthetic or non-naturally occurring amino acids refer to amino acids which do not naturally occur in vivo but which, nevertheless, can be incorporated into the peptide structures described herein. The resulting “synthetic peptide” contains amino acids other than the 20 naturally occurring, genetically encoded amino acids at one, two, or more positions of the peptides. For instance, naphthylalanine can be substituted for trytophan to facilitate synthesis. Other synthetic amino acids that can be substituted into peptides include L-hydroxypropyl, L-3,4-dihydroxyphenylalanyl, alpha-amino acids such as L-alpha-hydroxylysyl and D-alpha-methylalanyl, L-alpha.-methylalanyl, beta.-amino acids, and isoquinolyl. D amino acids and non-naturally occurring synthetic amino acids can also be incorporated into the peptides. Other derivatives include replacement of the naturally occurring side chains of the 20 genetically encoded amino acids (or any L or D amino acid) with other side chains.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

As used herein, the term “antibody” refers to a polyclonal or monoclonal antibody or a binding fragment thereof such as Fab, F(ab′)2 and Fv fragments.

As used herein, the term “SLLP polypeptide” refers to an amino acid sequence that comprises a sequence selected from the group consisting of SEQ ID NO: 16, SEQ ID NO: 17, SEQ ID NO: 18, SEQ ID NO: 19, SEQ ID NO: 20 and SEQ ID NO: 21.

As used herein, the term “SLLP antibody” refers to an antibody that specifically binds to an amino acid sequence selected from the group consisting of SEQ ID NO: 2, SEQ ID NO: 4, SEQ ID NO: 6, SEQ ID NO: 8, SEQ ID NO: 10, or SEQ ID NO: 12.

As used herein, the term “biologically active fragments” or “bioactive fragment” of an SLLP polypeptide encompasses natural or synthetic portions of the full-length protein that are capable of specific binding to their natural ligand.

The term “non-native promoter” as used herein refers to any promoter that has been operably linked to a coding sequence wherein the coding sequence and the promoter are not naturally associated (i.e. a recombinant promoter/coding sequence construct).

As used herein, a transgenic cell is any cell that comprises a nucleic acid sequence that has been introduced into the cell in a manner that allows expression of a gene encoded by the introduced nucleic acid sequence.

As used herein, the term “treating” includes alleviating the symptoms associated with a specific disorder or condition and/or preventing or eliminating said symptoms. For example, treating cancer includes preventing or slowing the growth and/or division of cancer cells as well as killing cancer cells.

### Embodiments

Six human sperm proteins have recently been isolated (SLLP1-6) that are testis specific and appear to be lysozyme-C paralogues. These proteins are classified as lysozyme paralogues because of their high degree of conservation of critical amino acids found in other lysozyme-C's. However, they differ significantly from the known human lysozyme-C in nucleic acid and amino acid sequence, and their genes are located on different chromosomes.

Of all known lysozyme-C sequences (>75), 20 amino acid residues are invariant. SLLP1 contains all but two of those invariable amino acids (E35T, D52N). The amino acid 35-E is considered a critical amino acid for catalytic function (i.e. cleaving the polysaccharide bond between N-actetylglucosamine and N-acetylmuramic acid). SLLP2 contains all but one (D52E) of the 20 conserved amino acids. The amino acid 53-L in humans is considered a critical amino acid for catalytic function; however, g-type lysozymes do not have a D in the corresponding position. Homologous genes of SLLP1 and SLLP2 have also been isolated by applicants from other mammalian species (for example, mice), that contain similar mutations in the catalytic residues of these genes.

The SLLP1 and SLLP2 proteins are approximately 15 kDa with pI's of 5.2 and 5.9, respectively. The proteins are expressed with an N-terminus signal peptide that is subsequently cleaved. The full length and mature forms of SLLP1 and SLLP2 are provided as SEQ ID NOs: 2 & 16 and SEQ ID NOs: 4 & 17, respectively. Both proteins possess sequence homology to the known human lysozyme-C; however, SLLP1 and SLLP2 are located on chromosome 17 and the X-chromosome, respectively, and thus these two genes represent new human lysozyme-like genes.

Recombinant SLLP1 and SLLP2 have been expressed in E. coli and in yeast. The proteins expressed in yeast were produced in a form that is secreted into the medium, and was purified from the media and used in an assay to test for lysozyme activity. Isolated putatively processed forms of SLLP1 and SLLP2 (SLLP2 was in crude form) from Pichia pastoris revealed no lysozyme activity for SLLP1 and SLLP2 using Micrococcus lysodeikticus as the lysozyme substrate. In particular, Micrococcus lysodeikticus was grown to confluence on a petri plate and the cells were contacted with 330 U of human lysozyme C (as a positive control), a reagent blank (as a negative control) and 1650 U of the purified soluble SLLP1 protein (yrSLLP1). Lysozyme activity was observed in the human lysozyme C portion (the positive control) as indicated by a zone of clearance about the introduce sample, but no activity was detected for yrSLLP1 or yrSLLP2. Similarly, no lysozyme activity was detected for E. coli synthesized SLLP1 or SLLP2.

Similarly, SLLP3 was also found to lack lysozyme activity based on expression from an E. coli system. SLLP3 is approximately 15.0 kDa, with a pI of 5.4 and located on chromosome 17 at locus 17q11.2. SLLP3 is expressed with an N-terminus signal peptide that is subsequently cleaved. The full length and mature forms of SLLP3 are provided as SEQ ID NOs: 6 & 18, respectively. SLLP3 shares amino acid sequence homology of 44% to human lysozyme; 45% to SLLP1; 47% to SLLP2 and contains all 20 conserved amino acids including both catalytic amino acids. Although recombinant SLLP1, SLLP2 and SLLP3 fail to exhibit lysozyme activity in the present assay, these compounds may still exhibit antibacterial/antiviral activity through an unknown mechanism.

SLLP4 is approximately 14.8 kDa, with a pI of 8.4 and located on chromosome 10 at locus 10p12. 1. SLLP4 is expressed with an N-terminus signal peptide that is subsequently cleaved. The full length and mature forms of SLLP4 are provided as SEQ ID NOs: 8 & 19, respectively. SLLP4 shares amino acid sequence homology of 48% to human lysozyme; 47% to hSLLP1 & 2; 42% to hSLLP3 and contains all 20 conserved amino acids including both catalytic amino acids. SLLP5 is approximately 14.8 kDa, with a pI of 8.4 and located on chromosome 10 at locus 10p11.23. SLLP5 is expressed with an N-terminus signal peptide that is subsequently cleaved. The full length and mature forms of SLLP5 are provided as SEQ ID NOs: 10 & 20, respectively. SLLP5 shares amino acid sequence homology of 48% to human Lysozyme; 46% to hSLLP1; 47% to hSLLP2; 43% to hSLLP3; 97% to hSLLP4 and contains all 20 conserved amino acids including both catalytic amino acids. SLLP6 is approximately 14.6 kDa, with a pI of 8.4 and located on chromosome 3 at locus 3p21.33. SLLP6 is expressed with an N-terminus signal peptide that is subsequently cleaved. The full length and mature forms of SLLP6 are provided as SEQ ID NOs: 12 & 21, respectively. SLLP6 shares amino acid sequence homology of 40% to human lysozyme; 49% to SLLP1; 40% to SLLP2; 48% to SLLP3; 41% to SLLP4; 42% to SLLP5 and contains 16 out of 20 amino acids including only one of the two catalytic amino acids. SLLP4, SLLP5 and SLLP6 have not been tested for lysozyme activity.

In accordance with one embodiment of the present invention a purified polypeptide is provided comprising the amino acid sequence of SEQ ID NO: 6, SEQ ID NO: 8, SEQ ID NO: 10 or SEQ ID NO: 12, or an amino acid sequence that differs from any of those sequences by one or more conservative amino acid substitutions. In another embodiment the purified polypeptide comprises an amino acid sequence that differs from SEQ ID NO: 6,SEQ ID NO: 8,SEQ ID NO: 10 or SEQ ID NO:12 by less than 5 conservative amino acid substitutions, and in a further embodiment, by 2 or less conservative amino acid substitutions. In accordance with one embodiment of the present invention a purified polypeptide is provided that consists of the amino acid sequence of SEQ ID NO: 18, SEQ ID NO: 19, SEQ ID NO: 20 or SEQ ID NO: 21, or a fragment of SEQ ID NO: 18, SEQ ID NO: 19, SEQ ID NO: 20 or SEQ ID NO: 21, or an amino acid sequence that differs from SEQ ID NO: 18, SEQ ID NO: 19, SEQ ID NO: 20 or SEQ ID NO: 21 by one to ten conservative amino acid substitutions.

The polypeptides of the present invention may include additional amino acid sequences to assist in the stabilization and/or purification of recombinantly produced polypeptides. These additional sequences may include intra- or inter-cellular targeting peptides or various peptide tags known to those skilled in the art. In one embodiment, the purified polypeptide comprises an amino acid sequence selected from the group consisting of SEQ ID NO: 2, SEQ ID NO: 4, SEQ ID NO: 6, SEQ ID NO: 8, SEQ ID NO: 10 and SEQ ID NO: 12 and a peptide tag, wherein the peptide tag is linked to the SLLP peptide sequence. Suitable expression vectors for expressing such fusion proteins and suitable peptide tags are known to those skilled in the art and commercially available. In one embodiment the tag comprises a His tag.

In another embodiment, the present invention is directed to a purified polypeptide that comprises an amino acid fragment of a SLLP polypeptide. More particularly the SLLP polypeptide fragment consists of natural or synthetic portions of a full-length polypeptide selected from the group consisting of SEQ ID NO: 6, SEQ ID NO: 8, SEQ ID NO: 10 and SEQ ID NO: 12 that are capable of specific binding to their natural ligand. Alternatively the fragment may comprise an antigenic fragment of a polypeptide selected from the group consisting of SEQ ID NO: 6, SEQ ID NO: 8, SEQ ID NO: 10 and SEQ ID NO: 12.

In one embodiment a purified polypeptide is provided comprising an amino acid sequence selected from the group consisting of SEQ ID NO: 18, SEQ ID NO: 19, SEQ ID NO: 20, SEQ ID NO: 21, and bioactive fragments thereof, wherein the amino acid sequence is conjugated to a hydrophobic compound. In one embodiment the hydrophobic compound is selected from the group consisting of a fatty acid glyceride, and a sorbitan fatty acid ester. In one embodiment the amino acid sequence of SEQ ID NO: 18, SEQ ID NO: 19, SEQ ID NO: 20, SEQ ID NO: 21, or bioactive fragment thereof, is conjugated to a mono- or di-ester of glycerol or fatty acid having 6 to 12 carbon atoms. Specific examples of such glycerols and fatty acids are caproic acid mono- or di-glyceride, caprylic acid mono- or di-glyceride, capric acid mono- or di-glyceride, and lauric acid mono- or di-glyceride.

In accordance with one embodiment a composition is provided for inducing an immune response against the SLLP polypeptides. In one embodiment the composition comprises a purified peptide that consists of an amino acid sequence selected from the group consisting of SEQ ID NO: 6, SEQ ID NO: 8, SEQ ID NO: 10, SEQ ID NO: 12, and an antigenic fragments of those sequences. In one embodiment the peptide consists of a sequence selected from the group of SEQ ID NO: 18, SEQ ID NO: 19, SEQ ID NO: 20 and SEQ ID NO: 21. The compositions can be combined with a pharmaceutically acceptable carrier or adjuvant and administered to a mammalian species to induce an immune response.

The present invention also encompasses nucleic acid sequences that encode human SLLPs. In one embodiment a nucleic acid sequence is provided comprising the sequence of SEQ ID NO: 1, SEQ ID NO: 3, SEQ ID NO: 5, SEQ ID NO: 7, SEQ ID NO: 9, SEQ ID NO: 11 or fragments thereof. In another embodiment a purified nucleic acid sequence is provided, selected from the group consisting of SEQ ID NO: 5 SEQ ID NO: 7, SEQ ID NO: 9 and SEQ ID NO: 11.

The present invention is also directed to recombinant human SLLP gene constructs. In one embodiment, the recombinant gene construct comprises a non-native promoter operably linked to a nucleic acid sequence selected from the group consisting of SEQ ID NO: 1, SEQ ID NO: 3, SEQ ID NO: 5, SEQ ID NO: 7, SEQ ID NO: 9, SEQ ID NO: 11 or fragments thereof. In another embodiment a non-native promoter is operably linked to a nucleic acid sequence selected from the group consisting of SEQ ID NO: 5, SEQ ID NO: 7, SEQ ID NO: 9 and SEQ ID NO: 1. The non-native promoter is preferably a strong constitutive promoter that allows for expression in a predetermined host cell. These recombinant gene constructs can be introduced into host cells to produce transgenic cell lines that synthesize the SLLP gene products. Host cells can be selected from a wide variety of eukaryotic and prokaryotic organisms, and two preferred host cells are E. coli and yeast cells.

In accordance with one embodiment, a nucleic acid sequence selected from the group consisting of SEQ ID NO: 5, SEQ ID NO: 7, SEQ ID NO: 9 and SEQ ID NO: 11 is inserted into a eukaryotic or prokaryotic expression vector in a manner that operably links the gene sequences to the appropriate regulatory sequences, and human SLLP is expressed in a eukaryotic or prokaryotic host cell. Suitable eukaryotic host cells and vectors are known to those skilled in the art. The baculovirus system is also suitable for producing transgenic cells and synthesizing the SLLP genes of the present invention. One aspect of the present invention is directed to transgenic cell lines that contain recombinant genes that express human SLLP and fragments of the human SLLP coding sequence. As used herein a transgenic cell is any cell that comprises an exogenously introduced nucleic acid sequence.

In one embodiment the introduced nucleic acid is sufficiently stable in the transgenic cell (i.e. incorporated into the cell's genome, or present in a high copy plasmid) to be passed on to progeny cells. The cells can be propagated in vitro using standard cell culture procedure, or in an alternative embodiment, the host cells are eukaryotic cells and are propagated as part of a plant or an animal, including for example, a transgenic animal. In one embodiment the transgenic cell is a human cell and comprises a nucleic acid sequence selected from the group consisting of SEQ ID NO: 5, SEQ ID NO: 7, SEQ ID NO: 9 and SEQ ID NO: 11. The present invention also includes non-human transgenic organisms wherein one or more of the cells of the transgenic organism comprise a recombinant gene that expresses a human SLLP product.

The present invention also encompasses a method for producing human SLLPs. The method comprises the steps of introducing a nucleic acid sequence, comprising a promoter operably linked to a sequence selected from the group consisting of SEQ ID NO: 1, SEQ ID NO: 3, SEQ ID NO: 5, SEQ ID NO: 7, SEQ ID NO: 9 and SEQ ID NO: 11, into a host cell, and culturing the host cell under conditions that allow for expression of the introduced human SLLP gene. In one embodiment the promoter is a conditional or inducible promoter, alternatively the promoter may be a tissue specific or temporal restricted promoter (i.e. operably linked genes are only expressed in a specific tissue or at a specific time). The synthesized SLLPs can be purified using standard techniques, and used in high throughput screens to identify inhibitors of SLLP activity. Alternatively, in one embodiment the recombinantly produced SLLP polypeptides, or fragments thereof are used to generate antibodies against the SLLP polypeptides. The recombinantly produced SLLP proteins can also be used to obtain crystal structures. Such structures would allow for crystallography analysis that would lead to the design of specific drugs to inhibit SLLP function.

Preferably, the nucleic acid sequences encoding the SLLPs are inserted into a suitable expression vector in a manner that operably links the gene sequences to the appropriate regulatory sequences for expression in the preselected host cell. Suitable host cells, vectors and methods of introducing the DNA constructs into cells are known to those skilled in the art. In particular, nucleic acid sequences encoding the SLLP proteins may be added to a cell or cells in vitro or in vivo using delivery mechanisms such as liposomes, viral based vectors, or microinjection.

Another embodiment of the present invention is directed to antibodies specific for one or more mammalian SLLPs. Antibodies to human SLLPs may be generated using methods that are well known in the art. In accordance with one embodiment an antibody is provided that binds to a polypeptide selected from the group consisting of SEQ ID NO: 6, SEQ ID NO: 8, SEQ ID NO: 10, and SEQ ID NO: 12. In one embodiment the antibody specifically binds to the polypeptides SEQ ID NO: 8 and SEQ ID NO: 10. In one embodiment the antibody specifically binds to the peptide sequence of SEQ ID NO: 6. In one embodiment the antibody specifically binds to the peptide sequence of SEQ ID NO: 8. In one embodiment the antibody specifically binds to the peptide sequence of SEQ ID NO: 10. In one embodiment the antibody specifically binds to the peptide sequence of SEQ ID NO: 12. In one embodiment the antibody is a monoclonal antibody. The antibodies may be used with or without modification, and may be labeled by joining them, either covalently or non-covalently, with a reporter molecule. In addition, the antibodies can be formulated with standard carriers and optionally labeled to prepare therapeutic or diagnostic compositions.

The antibodies or antibody fragments of the present invention can be combined with a carrier or diluent to form a composition. In one embodiment, the carrier is a pharmaceutically acceptable carrier. Such carriers and diluents include sterile liquids such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose, and related sugar solution, and glycols such as, propylene glycol or polyethylene glycol, are preferred liquid carriers, particularly for injectable solutions.

The present invention also provides a method for detecting the presence of human SLLP. The method comprises the steps of contacting a sample with a labeled antibody that specifically binds to human SLLP, removing unbound and non-specific bond material and detecting the presence of the labeled antibody. In one embodiment the labeled compound comprises an antibody that is labeled directly or indirectly (i.e. via a labeled secondary antibody). In particular, the SLLP antibodies of the present invention can be used to confirm the expression of SLLP as well as its cellular location, or in assays to monitor SLLP levels in individuals receiving a SLLP inhibitory composition as a means of contraception.

Northern blot analysis of SLLP1 & 2 indicated that these proteins are highly testis abundant, if not exclusively produced in the testis. To further characterize the expression of SLLP1 and SLLP2, antibodies were generated against SLLP1 and SLLP2. Those antibodies are specific for the target peptide and do not cross react with each other's respective lysozyme-like protein. SLLP1 immunofluorescence and SLLP1 and SLLP2 EM localization experiments demonstrate that expression of the SLLP1 and SLLP2 proteins are localized in the sperm acrosome. Furthermore, based on EST data, SLLPs 3-6 also appear to be primarily expressed in the testis. More particularly, Blast searches of the SLLP amino acid sequences revealed EST sequences primarily from the testis (i.e. for SLLP3: 21 testis and 1 medulla; SLLP4: 15 testis and 1 medula; SLLP5: 16 testis, 16 germ cell tumors and 5 other tumorous tissues; SLLP6: 8 testis, 9 germ cell tumors and single ESTs from medulla, retina and spleen). This makes the SLLP proteins optimal targets for the development of drugs that modulate their activity to determine their role in the fertilization process.

The SLLP polypeptides are anticipated to have sperm specific functions, and thus they are anticipated to have use in contraceptive compositions and/or serve as targets for the generation of contraceptive agents. In one embodiment, compositions comprising a SLLP polypeptide or fragments are administered to provide a contraceptive effect either directly or through the induction of an immune response. For example, in accordance with one embodiment the compositions comprising one or more of the individual SLLP polypeptides or antigenic fragments thereof are delivered to a subject to elicit an active immune response. The immune response generated in response to the antigenic composition acts as a temporary and reversible antagonist of the function of the SLLP proteins of the invention. Such vaccines can be used for active immunization of a subject, to raise an antibody response to temporarily block the sperm's access to the egg plasma antigen. In one aspect of the invention, an SLLP epitope can be administered at a certain period of the month, for example during ovulation of a female subject to block fertilization. Alternatively, contraception may be effected through passive immunity by the administration of compositions comprising antibodies specific for one or more of the SLLP polypeptides.

In another aspect of the invention, SLLP polypeptides (either separately or in combination) are used to elicit a T-cell mediated attack on the egg or sperm, having an othoritic effect, useful as a method for irreversible sterilization. Methods for generating T-cell specific responses, such as adoptive immunotherapy, are well known in the art (see, for example, Vaccine Design, Michael F. Powell and Mark J. Newman Eds., Plenum Press, New York, 1995, pp. 847-867). Such techniques may be particularly useful for veterinary contraceptive or sterilization purposes, where a single dose vaccination may be desirable.

The present invention also encompasses small molecule inhibitors of SLLP function and their use as contraceptive agents. In accordance with one aspect of the present invention, the SLLP family is used as a target for the development of novel drugs, and in one embodiment, compounds that specifically inhibit SLLPs from binding to their native ligands. Progress in the field of small molecule library generation, using combinatorial chemistry methods coupled to high-throughput screening, has accelerated the search for ideal cell-permeable inhibitors. In addition, structural-based design using crystallographic methods has improved the ability to characterize in detail ligand-protein interaction sites that can be exploited for ligand design.

In one embodiment, the present invention provides methods of screening for agents, small molecules, or proteins that interact with polypeptides comprising a sequence selected from the group consisting of SEQ ID NO: 6, SEQ ID NO: 8, SEQ ID NO: 10, and SEQ ID NO: 12 or bioactive fragments thereof. In another embodiment, the present invention provides methods of screening for agents, small molecules, or proteins that interact with polypeptides consisting of the sequence SEQ ID NO: 18, SEQ ID NO: 19, SEQ ID NO: 20, SEQ ID NO: 21, or bioactive fragments of such sequences. As used herein, the term “biologically active fragments” or “bioactive fragment” of SLLP polypeptides encompasses natural or synthetic portions of the native peptides that are capable of specific binding to at least one of the natural ligands of the respective native SLLP polypeptides. The invention encompasses both in vivo and in vitro assays to screen small molecules, compounds, recombinant proteins, peptides, nucleic acids, antibodies etc. which bind to or modulate the activity of SLLP and are thus useful as therapeutic or diagnostic markers for fertility. As used herein, modulating the activity of an SLLP includes interfering or altering the SLLPs ligand binding properties.

In one embodiment of the present invention SLLP polypeptides, selected from the group consisting of SEQ ID NO: 6, SEQ ID NO: 8, SEQ ID NO: 10, and SEQ ID NO: 12, are used to isolate ligands that bind to SLLP proteins under physiological conditions. The screening method comprises the steps of contacting a SLLP polypeptide with a mixture of compounds under physiological conditions, removing unbound and non-specifically bound material, and isolating the compounds that remain bound to the SLLP polypeptide. Typically, the SLLP polypeptide will be bound to a solid support, using standard techniques, to allow for rapid screening of compounds. The solid support can be selected from any surface that has been used to immobilize biological compounds and includes but is not limited to polystyrene, agarose, silica or nitrocellulose. In one embodiment the solid surface comprises functionalized silica or agarose beads. Screening for such compounds can be accomplished using libraries of pharmaceutical agents and standard techniques known to the skilled practitioner.

Ligands that bind to the SLLP polypeptides can then be further analyzed to determine if they interfere with sperm/oocyte binding using the binding and fusion assay described in Example 2 and Example 3. Inhibitors of SLLP activity have potential use as contraceptive agents. Such inhibitors can be formulated as pharmaceutical compositions and administered to a subject to block sperm/egg binding and fusion and thus provide a means for contraception.

In accordance with one embodiment of the present invention a method of decreasing mammalian sperm binding and fusion to mammalian oocytes is provided, wherein the activity of the SLLP proteins is inhibited, including for example, the binding activity of SLLPs with their natural ligands. In one embodiment the mammal is a human and the pharmaceutical composition comprises an inhibitor of SLLP activity. The inhibitor may constitute an antibody, small molecule antagonist, or the SLLP polypeptide itself, as well as compounds or nucleotide constructs that inhibit expression of the SLLP polypeptides (including but not limited to transcription factor inhibitors, antisense and ribozyme molecules, or gene or regulatory sequence replacement constructs).

In one embodiment a composition that inhibits sperm/egg binding and or fusion is provided that comprises an antisense or interference RNA that prevents or disrupts the expression of the SLLP genes in an animal. Interference RNA in mammalian systems includes the presence of short interfering RNA (siRNA), which consists of 19-22 nt double-stranded RNA molecules, or shRNA, which consists of 19-29 nt palindromic sequences connected by loop sequences. Down regulation of gene expression is achieved in a sequence-specific manner by pairing between homologous siRNA and target RNA. A system for the stable expression of siRNA or shRNA was utilized to generate transgenic animals (Hasuwa et al. FEBS Lett 532, 227-30 (2002), Rubinson et al. Nat Genet 33, 401-6 (2003) and Carmell et al. Nat Struct Biol 10, 91-2 (2003)) and can be used in accordance with the present invention to produce animals whose fertility can be regulated. A conditional RNAi-based transgenic system would provide the additional benefit of being able to control the level of gene expression at any given stage during the life of the animal.

In another embodiment a composition that inhibits sperm/egg binding and or fusion is provided that comprises an antibody against one or more of the SLLPs. In accordance with one embodiment antibodies are provided that specifically bind to all six SLLP polypeptides. Alternatively the composition may comprise an antibody that is specific for one or two of the individual SLLP polypeptides. Alternatively, in one embodiment an antibody is provided that specifically binds to the SLLP3, SLLP4, SLLP5 and SLLP6 polypeptides (i.e. the amino acid sequences of SEQ ID NOs: 6, 8, 10 and 12) but not to SLLP1 or SLLP2 (i.e. the amino acid sequences of SEQ ID NOs: 2 and 4). In a firer embodiment an antibody is provided that specifically binds to SLLP4 and SLLP5. In another embodiment an antibody is provided that specifically binds to SLLP3. In another embodiment an antibody is provided that specifically binds to SLLP6. In one embodiment, a SLLP3, SLLP4, SLLP5 or SLLP6 polypeptide, fragments thereof, or other derivatives, or analogs thereof, is used as an immunogen to generate antibodies which immunospecifically bind such an immunogen. In accordance with one embodiment of the preset invention an antigenic compound is provided for generating antibodies, wherein the compound comprises an amino acid sequence (or antigenic fragment thereof) selected from the group consisting of SEQ ID NO: 6, SEQ ID NO: 8, SEQ ID NO: 10 and SEQ ID NO: 12. The antibodies generated can be formulated with standard carriers and optionally labeled to prepare therapeutic or diagnostic compositions.

Antibodies to SLLP polypeptides or peptide fragments thereof may be generated using methods that are well known in the art. For the production of antibodies, various host animals, including but not limited to rabbits, mice, rats, etc. can be immunized by injection with a SLLP polypeptide or peptide fragment thereof. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum.

For preparation of monoclonal antibodies, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). In an additional embodiment of the invention, monoclonal antibodies can be produced in germ-free animals utilizing recent technology (PCT/US90/02545). According to the invention, human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). In fact, according to the invention, techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for epitopes of SLLP polypeptides together with genes from a human antibody molecule of appropriate biological activity can be used; such antibodies are within the scope of this invention.

According to the invention, techniques described for the production of single chain antibodies (U.S. Pat. No. 4,946,778) can be adapted to produce SLLP protein-specific single chain antibodies. An additional embodiment of the invention utilizes the techniques described for the construction of Fab expression libraries (Huse et al., 1989, Science 246:1275-1281) to allow rapid and easy identification of monoclonal Fab fragments with the desired specificity for egg surface proteins, derivatives, or analogs.

Antibody fragments which contain the idiotype of the molecule can be generated by known techniques. For example, such fragments include but are not limited to: the F(ab′)₂ fragment which can be produced by pepsin digestion of the antibody molecule; the Fab′ fragments which can be generated by reducing the disulfide bridges of the F(ab′)2 fragment, the Fab fragments which can be generated by treating the antibody molecule with papain and a reducing agent, and Fv fragments.

In the production of antibodies, screening for the desired antibody can be accomplished by techniques known in the art, e.g. ELISA (enzyme-linked immunosorbent assay). The foregoing antibodies can be used in methods known in the art relating to the localization and activity of the SLLP polypeptides of the invention, e.g., for imaging these proteins, measuring levels thereof in appropriate physiological samples, in diagnostic methods, etc.

Antibodies generated in accordance with the present invention may include, but are not limited to, polyclonal, monoclonal, chimeric (i.e. “humanized” antibodies), single chain (recombinant), Fab fragments, and fragments produced by a Fab expression library. These antibodies can be used as diagnostic agents for the diagnosis of conditions or diseases characterized by expression or overexpression of SLLP polypeptides, or in assays to monitor patients being treated with SLLP polypeptides receptor agonists, antagonists or inhibitors. The antibodies useful for diagnostic purposes may be prepared in the same manner as those described above for therapeutics. The antibodies may be used with or without modification, and may be labeled by joining them, either covalently or non-covalently, with a reporter molecule.

In one embodiment antibodies against the SLLP polypeptides are used as contraceptive agents that prevent the binding of sperm cells to eggs. An experiment was conducted to determine if antibodies against SLLP1 and SLLP2 could interfere with human sperm's ability to bind to eggs (See Example 2). The assay was conducted in vitro using human sperm and hamster eggs. SLLP1 and SLLP2 are on the acrosome membrane and are only exposed upon permeablization of the acrosome. Only approximately ⅓ of sperm undergo acrosome reaction in vitro. As seen in Example 2, antibodies against SLLP1 significantly interfered with sperm cells ability to bind to hamster eggs. No effect was observed for the antibody generated against SLLP2. These results suggest that a unique receptor for the SLLP1 protein may exist on mammalian eggs, and this receptor itself could serve as a target for contraceptive agents.

Recombinant human SLLP1 has been expressed in yeast and E. coli, and the recombinant proteins have been found to bind to the perivitelline matrix of both mouse and hamster eggs. Recombinant human lysozyme has also been expressed in E. coli, but this protein fails to bind to mouse eggs. Accordingly, the SLLP1 binding to the eggs appears to be a specific interaction that remains even after physical removal of the zona pellucida. The discovery that a processed form of SLLP1 in the acrosome of human spermatozoa with a similar c lysozyme like sequence and organization including retention of putative substrate binding residues conserved across human, mouse and rat orthologues leads to the hypothesis that this molecule may function as a potential receptor for the saccharide, N-acetylglucosamine. N-acetylglucosamine has been found in the extra cellular matrix over the egg plasma membrane, within the perivitelline space, pores of zona pellucida and in cumulus layers. Blocking the natural interaction between one or more of the SLLP polypeptides and their natural ligand(s) is anticipated to provide an effective means of contraception. Furthermore, due to the cross-species binding of the human SLLPs to other species, it is anticipated that a human SLLP based contraceptive composition will also be an effective contraceptive for other non-human mammalian species. Therefore, the SLLP based contraceptives may be used to prevent pregnancies in humans as well as in veterinarian species and more particularly in domesticated mammalian species including livestock.

One embodiment of the present invention is directed to compositions that can be placed in contact with sperm or oocytes to inhibit the function of the SLLP polypeptides (i.e. either by inhibiting the expression of the SLLP1, SLLP2, SLLP3, SLLP4, SLLP5, SLLP6 proteins or by interfering with the protein's function) or the natural ligand/receptor of those proteins. In one embodiment the SLLP inhibiting composition comprises peptide fragments of SLLP polypeptides, or analogs thereof that are taken up by sperm or egg and compete for binding with SLLP polypeptides' natural ligands. In accordance with one embodiment the exogenously added SLLP peptides interfere with the natural SLLPs' role in binding and fusion of the sperm and egg. Accordingly, compositions comprising a SLLP polypeptide inhibitory agent can be used to modulate fertility of an individual, and in one embodiment, the inhibitory agents function as a male contraceptive pharmaceutical. In accordance with one embodiment a composition is provided that comprises an eight to fifteen amino acid sequence that is identical to an eight to fifteen contiguous amino acid sequence selected from the group consisting of SEQ ID NO: 6, SEQ ID NO: 8, SEQ ID NO: 10 or SEQ ID NO: 12, and a pharmaceutically acceptable carrier.

In one embodiment the composition is formulated to inhibiting binding of sperm to oocytes, said composition comprising a polypeptide, wherein said polypeptide comprises an amino acid sequence selected from the group consisting of SEQ ID NO: 2, SEQ ID NO: 4, SEQ ID NO: 6, SEQ ID NO: 8, SEQ ID NO: 10, SEQ ID NO: 12, and bioactive fragments of such sequences. More particularly, the composition inhibits the binding of a mammalian sperm to a mammalian egg and in one embodiment the composition is used to inhibit the binding of a human sperm to a human egg.

In one embodiment the sperm/egg interfering composition consists of a peptide fragment of an amino acid sequence selected from the group consisting of SEQ ID NO: 2, SEQ ID NO: 4, SEQ ID NO: 6, SEQ ID NO: 8, SEQ ID NO: 10 and SEQ ID NO: 12 or in one embodiment the composition consists of a peptide fragment of an amino acid sequence selected from the group consisting of SEQ ID NO: 18, SEQ ID NO: 19, SEQ ID NO: 20 and SEQ ID NO: 21. For example, the peptide fragment may represent an amino acid sequence, ranging from about 50 to about 100, about 4 to about 40, about 5 to about 20, about 6 to about 15 or about 8 to 12 amino acids in length, identical to a contiguous sequence contained within an amino acid sequence selected from the group consisting of SEQ ID NO: 2, SEQ ID NO: 4, SEQ ID NO: 6, SEQ ID NO: 8, SEQ ID NO: 10 and SEQ ID NO: 12. As used herein the term “peptide fragment” is intended to include amino acid subsets of the parent polypeptide prepared either as enzymatic fragments of the parent polypeptide, synthesized recombinant peptide fragments or as chemically synthesized polyamino acids. In one embodiment the composition comprises a polypeptide consisting of the amino acid sequence of SEQ ID NO: 18, SEQ ID NO: 19, SEQ ID NO: 20 or SEQ ID NO: 21. In another embodiment the composition comprises a polypeptide consisting of the amino acid sequence of SEQ ID NO: 2, SEQ ID NO 16, or a bioactive fragment of SEQ ID NO: 2. The composition comprising the amino acid sequence of SEQ ID NO: 2, or a fragment thereof, can be further combined with a second polypeptide selected from the group consisting of SEQ ID NO: 18, SEQ ID NO: 19, SEQ ID NO: 20, SEQ ID NO: 21 and peptide fragments of SEQ ID NO: 18, SEQ ID NO: 19, SEQ ID NO: 20, SEQ ID NO: 21.

The sperm/egg interfering composition will typically include a pharmaceutically acceptable carrier. In addition, the composition may also include stabilizing agents and agents that assist in the delivery of the peptides. In one embodiment the SLLP polpeptides or peptide fragments thereof are formulated to be delivered transdermally. In such formulations, the SLLP compositions may comprising a compound selected from the group consisting of propylene glycol, a monohydric alcohol having 2 to 4 carbon atoms, lactic acid, thioglycol, a fatty acid glyceride, and a sorbitan fatty acid ester. Such inhibitory peptides can also be modified to include fatty acid side chains to assist the peptides in penetrating the sperm cell and/or egg membranes.

A pharmaceutical composition for transdermal administration in accordance with this invention is generally prepared by dispersing a pharmacologically-active substance and other ingredients in a nontoxic, pharmaceutically acceptable liquid base to produce a suspension or gel. Techniques for preparing transdermal formulations are known to those skilled in the art as described in U.S. Pat. Nos. 6,106,856 and 4,637,930, the disclosures of which are incorporated herein. In one embodiment the SLLP polypeptides/peptides are combined with a compound selected from the group consisting of polyethylene glycol, cis-oleic acid, dimethylisosorbide and propylene glycol. The contribution of each component can be varied between 0.1 to 98 mole fraction percent. In one embodiment the formulation comprises between 1 to 10 mole percent cis-oleic acid and between 1 to 10 mole percent dimethylisosorbide dispersed in propylene glycol.

In an another embodiment a transdermal formulation is prepared comprising an SLLP polypeptide/peptide and a compound selected from the group consisting of propylene glycol, a monohydric alcohol having 2 to 4 carbon atoms, lactic acid, thioglycol, a middle chain fatty acid glyceride, and a sorbitan middle chain fatty acid ester and a urea, said liquid being in a transdermal formulation state. Examples of the monohydric alcohol having 2 to 4 carbon atoms for use in this invention are ethanol, propanol, isopropanol, etc. Examples of the middle chain fatty acid glyceride for use in this invention are mono- or di-esters of glycerol and fatty acids having 6 to 12 carbon atoms. Specific examples of such glycerols and fatty acids are caproic acid mono- or di-glyceride, caprylic acid mono- or di-glyceride, capric acid mono- or di-glyceride, and lauric acid mono- or di-glyceride. These materials may be used solely as a mixture of two or more materials. For example, a mixture of 54.3% caprylic acid mono-glyceride and 37% caprylic acid di-glyceride is commercially available as a trade name “Nikkol MGK” (made by Nikko Chemicals Co.) or a product containing more than 85% caprylic acid mono-glyceride is commercially available as a trade name “Sunsoft No. 700p-2” (made by Taiyo Kagaku K. K.). As the sorbitan middle chain fatty acid ester for use in this invention, there are mono- or di-esters of sorbitol and fatty acids of 6 to 12 carbon atoms. Specific examples of these esters are sorbitan mono-caproic acid ester, sorbitan dicaproic acid ester, sorbitan monocaprylic acid ester, sorbitan dicaprylic acid ester, sorbitan monocapric acid ester, sorbitan dicapric acid ester, sorbitan monolauric acid ester, and sorbitan dilauric acid ester. They can be used solely or as a mixture of them.

The SLLP containing compositions can also be formulated to be administered directly to the vagina. In accordance with one embodiment a self-administrable antifertility composition comprising an SLLP polypeptide for topical non-systemic application to the cervix and or vagina is provided. The composition comprises an amino acid sequence selected from the group consisting of SEQ ID NO: 2, SEQ ID NO: 4, SEQ ID NO: 6, SEQ ID NO: 8, SEQ ID NO: 10 and SEQ ID NO: 12 and a non-rigid pharmaceutically acceptable viscous gel, cream, foam or effervescent type suppository vehicle. Suitable gel, cream, foam and suppository delivery vehicles are well known to those skilled in the art and can be used to prepare the topical antifertility compositions of the present invention.

In another embodiment of the present invention the specific egg binding properties of the SLLP polypeptides allow them to be used for imaging the ovary and oocytes in vivo. In one embodiment the imaging composition comprises an amino acid sequence selected from the group consisting of SEQ ID NO: 2, SEQ ID NO: 4, SEQ ID NO: 6, SEQ ID NO: 8, SEQ ID NO: 10 and SEQ ID NO: 12, or a bioactive fragment thereof, wherein said amino acid sequence is labeled, either directly or indirectly with a detectable label. The term “label” as used herein refers to any atom or molecule which can be used to provide a detectable (preferably quantifiable) “signal”, and which can be attached to the protein. Labels may provide “signals” detectable by fluorescence, radioactivity, colorimetry, gravimetry, X-ray diffraction or absorption, magnetism, enzymatic activity, and the like. It is not intended that the present invention be limited to any particular detection system or label, other than requiring the lable to be detectable in vivo through the use of a non-invasive means. Such labels and detection systems are know to those skilled in the art. For example the polypeptides can be labeled with metal ion source such as gadolinium trichloride, dysprosium trichloride or a technicium or indium derivative.

In accordance with one embodiment imaging of oocytes throught the use of labeled SLLP polypeptides is used to quantitate the number of oocytes present in the ovaries of a female, or in another embodiment is used to detect ovarian cancer. The imaging of oocytes may be an important diagnostic tool for identifying patients that suffer from premature ovarian failure and thus will allow for treatment prior to the loss of all oocytes. In accordance with one embodiment a non-invasive method of imagining oocytes in vivo comprises the step of administering a composition comprising a labeled SLLP polypeptide to a female patient and then detecting the label after a predetermined length of time subsequent to the administration of the composition. In accodance with one embodiment the composition is administered vaginally as a topical agent, however other routes of administration, including injection directly into, or adjacent to, the ovaries, or transdermal administration, are known to those skilled in the art and can be used to administer the composition.

In another aspect of the present invention, since ESTs corresponding to SLLP5 and SLLP6 have been detected in tumorous tissues, somatic expression of these proteins may serve as a marker for neoplastic disease. Accordingly, one embodiment of the invention is directed to the use of SLLPs, and in particular SLLP5 and SLLP6, as diagnostic markers for neoplastic disease such as cancer. The method would comprise the steps of screening for elevated levels or inappropriate expression of SLLPs, including the expression of SLLPs in somatic tissues. Such screens could be conducted using antibodies specific for the SLLP polypeptides. Alternatively, antibodies directed against SLLP polypeptides can be used in assays to monitor patients being treated with anticancer therapies to monitor the effectiveness of the therapy.

### EXAMPLE 1

### Isolation of the SLLP1 and SLLP2 Proteins

Materials and Methods

Solubilization and Electrophoresis of Human Spermatozoal Proteins

Preparation of semen specimens and solubilization of sperm proteins were performed as previously described (Naaby-Hansen et al, Biol Reprod 1997; 56:771-787) For analytical two-dimensional electrophoresis the detergent/urea extracted proteins were separated by isoelectric focusing (IEF) in acrylamide tube gels prior to second dimensional gel electrophoresis (SDS-PAGE), which was performed in a Protean II xi Multi-Cell apparatus (Bio-Rad, Richmond, Calif.) or on large format (23×23 cm) gels (Investigator 2-D Electrophoresis System, ESA) which were also employed for preparative 2D gel electrophoresis. Electrotransfer to nitrocellulose membranes and subsequent visualizing of the proteins by gold staining was accomplished as previously described (Naaby-Hansen et al, 1997) while electrotransfer to PVDF membranes (0.2 mm pore size, Pierce) was carried out as described by Henzel et al. (1993) using the transfer buffer composition of Matsudaira (1987) (10 mM 3-[cyclohexylamino]-1-propanesulfonic acid, 10% methanol, pH 11). The immobilized proteins were visualized by staining in a solution containing 0.1% Commassie R250, 40% methanol and 0.1% acetic acid for one minute, followed by destaining in a solution of 10% acetic acid and 50% methanol for 3×3 minutes.

Microsequencing of the SLLP1 and SLLP2 Proteins

The SLLP1 and SLLP2 stained protein spots were cored from a 1.5 mm thick 2D SDS-polyacrylamide gel and fragmented into smaller pieces. The proteins were destained in methanol, reduced in 10 mM dithiothreitol and alkyiated in 50 mM iodoacetamide in 0.1 M ammonium bicarbonate. After removing the reagents, the gel pieces were incubated with 12.5 ng/ml trypsin in 50 mM ammonium bicarbonate overnight at 37° C. Peptides were extracted from the gel pieces in 50% acetonitrile in 5% formic acid and microsequenced by tandem mass spectrometry and by Edman degradation at the Biomolecular Research Facility of the University of Virginia. Differentiation of leucine and isoleucine in the sequences were determined by Edman sequencing of HPLC isolated peptides. A degenerate deoxyinosine containing primers were used to isolate the SLLP1 and SLLP2 cDNA clones based on the microsequencing data and using PCR technology.

Northern and Dot Blot Analyses

A Northern blot containing 2 mg of poly(A)⁺ RNA from eight selected human tissues was obtained from Clontech. The Northern blot was probed with a ³²P-labeled SLLP1 cDNA or ³²P-labeled SLLP2 cDNA. Probes were prepared by random oligonucleotide prime labeling (Feinberg and Vogelstein, 1983). Hybridization was performed in ExpressHyb solution (Clontech) at 68° C., for 1 h followed by three washes in 2×SSC, 0.05% SDS at room temperature and two washes in 0.1×SSC, 0.1% SDS for 20 min at 50° C.

A normalized RNA dot blot containing 89 to 514 ng of mRNA from 50 different human tissues was obtained from Clontech and probed with ³²P-labeled SLLP1 cDNA or ³²P-labeled SLLP2 cDNA. The normalized (100-500 ng) poly-(A)+ mRNAs present on the grid were isolated from various tissue sources including: whole brain, amygdala, caudate nucleus, cerebellum, cerebral cortex, frontal lobe, hippocampus, medulla oblongata, occipital lobe, putamen, substantia nigra, temporal lobe, thalamus, subthalmic nucleus, spinal chord, heart, aorta, skeletal muscle, colon, bladder, uterus, prostate, stomach, testis, ovary, pancreas, pituitary gland, adrenal gland, thyroid gland, salivary gland, mammary gland, kidney, liver, small intestine, spleen, thymus, peripheral leukocyte, lymph node, bone marrow, appendix, lung, trachea, placenta, fetal brain, fetal heart, fetal kidney, fetal liver, fetal spleen, fetal thymus, fetal lung, and 100 ng total yeast RNA, 100 ng yeast tRNA, 100 ng E. coli rRNA, 100 ng E. coli DNA, 100 ng poly r(A), 100 ng Cot 1 human DNA, 100 ng human DNA, 500 ng human DNA. The blot was hybridized in ExpressHyb solution (Clontech) containing salmon sperm DNA and human placental Cot-1 DNA overnight at 65° C. The blot was then washed three times in 2×SSC, 1% SDS at 65° C., followed by two additional washes in 0.1×SSC, 0.5% SDS at 55° C., before exposing the filter to X-Ray film. Hybridization was only detected in the testis RNA dot.

### EXAMPLE 2

### Human Sperm Binding and Fusion Assay Using Zona-Free Hamster Eggs

Sperm Preparation:

Motile human sperm were harvested by the swim up method of Bronson and Fusi (1990). Briefly, a 500 ml sperm sample underlaid in 2 ml of BWW media containing 5 mg/ml HSA. Sperm were allowed to swim up for 1.5-2 h. Swimup sperm were collected and 8 ml of BWW+5 mg/ml HSA was added. The composition was spun at 600×g for 8 min at RT, the supernatant was removed and 8 ml of media was added to the pellet. The resuspended pellet was spun at 600×g for 8 min at RT. The supernatant was removed and 50 ml of BWW containing 30 mg/ml HSA was added to the pellet. Total sperm cells were counted and then incubated overnight in BWW+30 mg/ml HSA at a concentration of 20×10⁶ sperm/ml.

Egg Collection:

Female hamsters received i.p. injections of 30 IU PMSG followed by 30 IU of hCG 72 h later. 14-16 h following hCG injection, hamsters were sacrificed and oviducts are collected in BWW media containing 5 mg/ml HSA. Cumulus cells were removed with 1 mg/ml hyaluronidase, the eggs were washed and zona pellucidae removed with 1 mg/ml trypsin. The eggs were then thoroughly washed and allowed to rest in the incubator.

Sperm/Antibody Incubation:

Sperm was diluted to 20×10⁶ sperm/ml and incubated with appropriate dilutions of pre-immune or immune sera (initially a 1:10 and 1:50 dilution of sera is tested) in paraffin oil covered microdrops for 1 h.

Hamster eggs were added to the drops containing the sperm+antibody. The gametes were then co-incubated for 3 h.

Assessment of Binding and Fusion:

Eggs were washed free of unbound and loosely bound sperm by serial passage through 5 (50 ml) wash drops. The same pipet is used for all eggs washed in an individual experiment. Eggs are then stained by short-term (5-15 s) exposure to 1 mM acridine orange-3% DMSO in BSA/BWW (30 mg/ml), washed through 4 (50 ml) wash drops and mounted under 22×22 mm coverslips. Under UV illumination, unexpanded heads of oolemma-adherant sperm were counted and sperm that had penetrated the ooplasm exhibited expanded green heads. All experiments were repeated 3 times

Results
1:10 dilution of hSLLP1 Antibody
Number of sperm bound per egg

| | | | |
|---|---|---|---|
| Pre Immune | 38.2 | Immune | 21.8 |

P value = 7.78 × 10⁶
Number of sperm fused per egg

| | | | |
|---|---|---|---|
| Pre Immune | 3.2 | Immune | 2.9 |

P value = 0.6
1:10 dilution of hSLLP2 Antibody
Number of sperm bound per egg

| | | | |
|---|---|---|---|
| Pre Immune | 28.7 | Immune | 27.4 |

P value = 0.79
Number of sperm fused per egg

| | | | |
|---|---|---|---|
| Pre Immune | 1.8 | Immune | 1.6 |

P value = 0.71

### EXAMPLE 3

### Isolation of the Mouse Orthologue of hSLLP1

To analyze the function of the c lysozyme-like SLLP proteins in fertilization the mouse SLLP orthologues were isolated and mouse fertilization experiments were conducted. The novel intra-acrosomal c lysozyme-like protein hSLLP1 possesses 17 out of the 20 invariant residues of the c lysozymes including the all 8 cysteines but lacked both catalytic residues. However, most of the potential residues required for substrate binding in c lysozymes remain in hSLLP1. The murine orthologue of hSLLP1 (mSLLP1) was cloned and localized in the male gamete before and after acrosome reaction. In vitro fertilization assays were performed in the presence of recmSLLP1 as well as monospecific antibodies against mSLLP1. Moreover, complementary binding sites for mSLLP1 were identified on the female gamete in the mouse.

### MATERIALS AND METHODS

Cloning and Expression of mSLLP1

Using a Blast search tool (Altschul et al. 1990), a mouse orthologue of the human SLLP1 was sought in the NCBI GenBank database and a candidate gene identified. Single gene-specific forward (5′CAT GCC ATG GCC AAG GTC TTC AGT CGC TGT GAG CTG; SEQ ID NO: 14) and reverse primers (5′CCG CTC GAG GAA GTC ACA GCC ATC CAC CCA GTC; SEQ ID NO: 15) with NcoI and XhoI restriction sites respectively were designed to amplify the predicted processed form (128 amino acids, from 94 to 221) of the mouse SLLP1. Primers were obtained from Invitrogen (Carlsbad, Calif.). The cDNA was amplified by PCR from a mouse testis cDNA library (Clontech, Palo Alto, Calif.). The cycling parameters employed were 94° C., 2 min; 94° C., 30 sec; 51° C., 1 min; and 68° C., 1.5 min, for 40 cycles. PCR reaction products were separated on agarose gels, and a band of ˜400 bp was isolated, reamplified and subcloned in pCR2.1 TOPO vector (Invitrogen). Multiple cDNA clones were sequenced in both directions using vector-derived primers on a Perkin-Elmer Applied Biosystems DNA sequencer (Biomolecular Research Facility, Univ. of Virginia Health System, Va.). The insert was then restriction digested, gel purified, ligated into the predigested pET28b+ vector and used to transform competent BL21DE3 cells (Novagen, Madison). The final construct added two amino acids at the N-terminus and eight residues at the C-terminus including a six histidine tag.

A 2L culture from a single colony was grown to optical density of ˜0.8 at 600 ₙₘ at 37° C., in Luria broth (LB) in the presence of 50 μg/ml of kanamycin. Isopropyl-β-D-thiogalactopyranoside (IPTG) (Sigma, St. Louis, Mo.) was then added to a final concentration of 1 mM to induce expression. Following 3 h of induction, the bacteria were collected by centrifugation. The recombinant protein was isolated from the insoluble fraction of E. coli, dissolved in 8 M urea in binding buffer (20 mM tris-HCl, pH 7.9, 5 mM imidazole and 0.5 M NaCl) and purified on a His binding Ni²⁺ chelation affinity resin column by a modification of the manufacture's procedures (Novagen, Madison, Wis.). The eluates were then dialyzed overnight against three changes of PBS. The dialized protein was stored at −20° C., until used. Protein concentrations were determined by Coomassie Plus-200 (Pierce, Rockford, Ill.) using bovine serum albumin (BSA) as a standard.

Polyclonal Antibody Production and Western Blot Analysis

Five adult virgin female guinea pigs were used for antibody production against the purified recmSLLP1. Preimmune serum was collected by heart puncture, and subsequently, each animal was injected with 200 μg of the purified recmSLLP1 in complete Freund's adjuvant and boosted twice at intervals of 14 days with the same amount of protein in incomplete Freund's adjuvant. For all immunizations, half of the antigen emulsion was injected intramuscularly in the legs and half subcutaneously in two sites on the back. All animals were then exsanguinated by heart puncture 9 days after the final immunization, and the blood was collected in serum separation tubes (Becton Dickinson, Franklin Lakes, N.J.). After centrifugation at 1750 g for 10 min, the serum was removed, aliquoted and frozen until needed.

Specificity of the antisera was tested against recmSLLP1 and mouse sperm extracts following ID SDS-PAGE western blotting. RecmSLLP1 (0.1 μg/lane) or cauda epididymal mouse spermatozoa (10 μg/lane) were solubilized in Laemmli buffer (2×) and proteins were resolved on a 15% SDS-PAGE gel and separated at 20 mA. Proteins were then blotted to nitrocellulose and stained by Ponceau. All blots were blocked with 5% nonfat dry milk in PBS with 0.05% Tween 20 (PBS-T) for 30 min at room temperature. For immunoblotting of recmSLLP1 purified protein, 1:15000 or 1:30000 dilution of the anti-recmSLLP1 guinea pig sera was tested, whereas for mouse sperm proteins, 1:5000 or 1:10000 dilution of the sera was used. The blots were then washed three times for 10 min in PBS-T, and incubated with 1:5000 dilution of peroxidase conjugated goat anti-guinea pig IgG secondary antibody for 1 h and washed two times for 10 min in PBS-T. The blots were developed with ECL reagent (Amersham, Corp., Buckinghamshire, UK) and then in TMB peroxidase substrate (3,3′,5,5′-tetramethylbenzidine, Kirkegaard and Perry Laboratories, Gaithersburg, Md.).

Culture Media and Reagents for in vitro Fertilization Assays

The medium used for in vitro fertilization assays was Fraser's modification of Whittingham's medium (Fraser and Drury, 1975) supplemented with 3% BSA and prepared with culture grade H₂O with analytical-grade reagents. TYH (Toyoda et al., 1971) medium was used for sperm-oolemma binding assays. Pregnant mare's serum gonadotrophin (PMSG), human chorionic gonadotrophin (hCG), BSA, culture grade H₂O, hyaluronidase, chymotrysin, Hoeschst dye 33342 and all other reagents were all obtained from Sigma.

Gamete Preparation for in vitro Assays

Hybrid F1 mice (CB57BL/6J×CBA) were used in all experiments. Suspensions of epididymal spermatozoa from sexually mature male mice were prepared for insemination of isolated oocytes. Oocytes were obtained from 28-day-old females superovulated with 10 IU PMSG and 10 IU hCG, injected intraperitoneally 48 h apart. Females were killed 16 h after hCG injection and both oviducts were immediately removed and placed in mineral oil.

In vitro Fertilization with Cumulus-Oocyte Complexes

In vitro fertilization with cumulus intact oocytes was conducted with sperm dispersed from cauda epididymides placed for 5 min in 200 μl drops of fertilization medium under paraffin oil. The sperm suspension was diluted to a concentration of 10⁶ sperm/ml in a volume of 200 μl and then incubated for 120 min in a humidified tissue culture incubator (37° C., 5% CO₂ in air) to allow capacitation. In the experiments where recmSLLP1 serum was tested, spermatozoa were incubated with varying concentrations of decomplemented immune or preimmune serum for the last 45 min of capacitation. In the experiments where recmSLLP1 was evaluated, spermatozoa were incubated under standard capacitating conditions.

Cumulus masses were placed in 135 μl drops of fertilization medium (one mass per drop) under paraffin oil and were incubated for 45 min with immune or preimmune serum or in the presence or absence of recmSLLP1 prior to insemination. Fifteen μl of the sperm suspension (final concentration: 10⁵ sperm/ml) was then added to each cumulus mass drop. Thus, sera or recombinant protein was present in the incubation droplet during gamete interaction. Six hours following insemination oocytes were relocated in 100 μl drops of fertilization medium under mineral oil. Following overnight incubation, eggs were stained in 10 μg/ml Hoeschst dye for 10 min and washed 3 times in fertilization medium. The eggs were then placed in 5 μl drop fertilization medium between a microscope slide and an elevated coverslip, and visualized at 160× using light and fluorescence microscopy (Zeiss Axioplan). Two cells embryos were scored as fertilized while one-celled oocytes were scored as unfertilized.

In vitro Fertilization with Zona-Free Eggs

For the sperm-oolemma binding assay, two cauda epididymides were placed in 900 μl drops of fertilization medium under paraffin oil and the dense mass of spermatozoa was allowed to flow freely for 3 h. Cumulus oocyte complexes were placed in 200 μl drops of TYH medium under paraffin oil. Cumulus cells were removed by treating the oocytes for 3 min with 1 mg/ml hyaluronidase in TYH medium and then washed 8 times in 50 μl drops. Zona pellucidae were loosened by treating the oocytes with 10 μg/ml chymotripsin in TYH media for 1 min and loosened zonae were removed by mechanical agitation using a pulled Pasteur pipette. The oocytes were then washed 10 times and allowed to recover from chymotrypsin treatment by incubating in TYH media for 3 h, following which, they were stained with 10 μg/ml Hoeschst dye for 10 min, and then gently washed.

In the experiments where anti-recmSLLP1 sera was tested, spermatozoa were incubated with varying concentrations of decomplemented immune or preimmune sera for the last 30 min of capacitation. Untreated oocytes were then added to the incubation drops containing the treated sperm with a final concentration of 2.5×10⁴ sperm/ml. In those experiments where either recmSLLP1 or lysozymes was evaluated, spermatozoa were incubated under standard capacitating conditions. Oocytes were pre-incubated for 45 min before insemination with recmSLLP1(0.1 to 200 μg/ml) or with chicken or human lysozymes (50 μg/ml, 100 μg/ml). Untreated sperm were then added to the incubation drops containing the treated eggs with a final concentration of 2.5×10⁴ sperm/ml. Thus, in all the experiments performed the sera, the recombinant protein or the lysozymes was present in the incubation droplet during gamete interaction. After 30 min of gamete co-incubation, oocytes were gently washed 5 times in TYH medium and placed between a microscope slide and an elevated coverslip and visualized at 160×. Binding to the oocyte was scored by counting the total number of bound spermatozoa per oocyte using phase contrast. Fusion with the egg was scored by counting the number of decondensed sperm heads within each oocyte using fluorescence microscopy.

Indirect Immunofluorescence Studies of Mouse Spermatozoa and Oocytes

Labeling on Fixed Spermatozoa:

Cauda epididymal mouse spermatozoa were placed in 0.9 ml drop of phosphate-buffered saline without calcium (PBS; pH=7.4) (two epididymidies per drop) and incubated at 37° C., in an atmosphere of 5% CO₂ for 5 min. To induce the acrosome reaction, spermatozoa were incubated in TYH media for 90 min and 5 μM calcium ionophore A23187 was added for another 15 min. Each drop was then collected, centrifuged for 10 min at 500 g and resuspended in PBS; this washing procedure was repeated three times. Smears of the final suspension of mouse sperm in PBS were air-dried on microscope slides at room temperature and fixed in 2% w/v paraformaldehyde in PBS for 10 min. After 6 washes in PBS spermatozoa were incubated for 30 min at 37° C., with normal goat serum (NGS) (5% v/v in PBS) and then incubated for 1 h with anti-recmSLLP1 IgG from guinea pig (200 μg/ml). The slides were washed 3 times in PBS and spermatozoa were incubated for 1 h at 37° C., with Texas red-conjugated polyclonal antibody from donkey (1:200, Jackson Laboratories). Slides were then washed, incubated for 30 min at room temperature with Peanut agglutinin lectin (PNA) (1:50) (Molecular Probes), washed, mounted in Slowfade® (Molecular Probes), and visualized under a Zeiss Standard 18 ultraviolet microscope. Images were captured by using MrGrab (Carl Zeiss Vision GmbH, Germany).

Egg Labeling:

Metaphase II eggs were obtained as previously described (Coonrod et al, 1999) and incubated with 5% NGS/media for 30 min. Oocytes were washed five times in TYH medium and incubated with 100 μg/ml recmSLLP1 or 100 μg/ml lysozymes for 45 min at 37° C., and 5% CO₂. Oocytes were washed five times and incubated with guinea pig anti-recmSLLP1 polyclonal antibody (1:50), sheep anti-human lysozyme (1:25) or rabbit anti-chicken lysozyme (1:400) in 5% NGS/media for 1 h at37° C., and 5% CO₂. Oocytes were washed five times and incubated with donkey anti-guinea pig/Texas Red antibody (1:200) or goat anti-guinea pig/FITC antisera (1:200), donkey anti-sheep and goat anti-rabbit FITC-labeled secondary antibody (1:200) (Jackson ImmunoResearch), respectively in 5% NGS/media for 1 h at room temperature at 37° C., and 5% CO₂. Oocytes were washed and mounted in media onto glass slides and visualized under a Zeiss Standard 18 ultraviolet microscope. Images were captured by using MrGrab 1.0 (Carl Zeiss Vision GmbH, Germany).

Scanning Confocal Microscopy

Egg Labeling:

Metaphase II eggs that were used for immunofluorescence studies were washed three times in PBS+1% BSA (PBS/BSA) and fixed in 4% paraformaldehyde in PBS-polyvinylalcohol (PVA) for 20 min at room temperature. Following fixation, eggs were washed 5 times in PBS/BSA and then permeabilized with 0.5% Triton X-100 in PBS for 20 min at room temperature. Eggs were then washed five times in PBS/BSA and placed in 0.4 mg/ml RNase in PBS/BSA for 30 min and then stained with 20 nM Sytox (Molecular Probes, Eugene, Oreg.) for 10 min. Eggs were then extensively washed, placed in slow fade (Molecular Probes, Eugene, Oreg.) equilibration media for approximately 1 min and then mounted on slides in slow fade mounting media. Images were obtained on a Zeiss 410 Axiovert 100 microsystems LSM confocal microscope. For each panel, attenuation, contrast, brightness and pinhole aperture remained constant. For each panel, four seconds scans were averaged four times per line using a 63× oil lens equipped with a zoom factor of two.

Sperm Labeling During Binding to Metaphase II Eggs:

Zona-free eggs inseminated with capacitated spermatozoa that were used in the in vitro fertilization studies were fixed with 2% paraformaldehyde for 10 min at room temperature. Gametes were washed in PBS-BSA, incubated with 5% NGS/PBS-BSA for 30 min at 37° C., and then incubated for 1 h with recmSLLP1 IgG (200 μg/ml). The slides were washed 3 times in PBS and gametes were incubated for 1 h at 37° C., with donkey anti-guinea pig texas red-conjugated polyclonal antibody (1:200, Jackson Laboratories). Gametes were then washed five times in PBS/BSA, placed in 0.4 mg/ml RNase in PBS/BSA for 30 min and then stained with 20 nM Sytox (Molecular Probes, Eugene, Oreg.) for 10 min. Gametes were extensively washed, placed in slow fade (Molecular Probes, Eugene, Oreg.) equilibration media for approximately 1 min and then mounted on slides in slow fade mounting media. Images were obtained on a Zeiss 410 Axiovert 100 Microsystems LSM confocal microscope as described above.

### RESULTS

Mouse SLLP1 is the True Orthologue of hSLLP1 and Shares Similar Characteristics to C Lysozymes

The complete deduced amino acid sequence of mSLLP1 is provided as SEQ ID NO: 13. The N terminus of mSLLP1 contains a predicted transmembrane domain followed immediately by a potential protease cleavage site between the alanine 93 and lysine 94 linkage. Comparison of the full-length hSLLP1 and mSLLP1 sequences using the Accelrys Gap (Seq/Web version 2) algorithm found that mSLLP1 is 64.2% similar and 58.8% identical to the hSLLP1. The predicted processed form of mSLLP1 starting immediately after the protease cleavage site (128 aa) shares 82.8% similarity and 75.8% identity to the hSLLP1 processed form. The deduced mSLLP1 sequence contains three putative myristoylation sites, potential phosphorylation sites for casein kinase II (S97) and protein kinase C (S66, S90, S152, and S153) and a signature sequence for the alpha-lactalbumin/lysozyme C family. Moreover, the predicted molecular weight (14 kDa) and pI (5.2) of mature mSLLP1 are similar to hSLLP1.

In addition, a Blast search in the GenBank database and a multiple alignment of selected mature c lysozymes revealed that mature mSLLP1 is 46%, 48% and 50% identical to mouse, human and chicken lysozymes, respectively. Alignment of the mature form of mSLLP1 with these three c lysozymes revealed the presence of 44 identical residues, although the essential catalytic residues (E35 and D52) of lysozymes were replaced with T35 and N52 in mSLLP1. Among the six potential substrate-binding residues of lysozymes (residues at positions 37, 63, 64, 102, 109 and 115), five were conserved in mSLLP1 suggesting that mSLLP1 may be closely related to c lysozymes.

Expression of mSLLP1 and Specificity of the Antibody

A CDNA sequence encoding the mature, processed mSLLP1 protein from residue 94 to 201 (lacking the signal peptide and putative transmembrane domain), but with a six-His N terminal tag was expressed in E. Coli and a recombinant protein of ˜15 kDa was obtained after Ni⁺⁺-affinity purification. To evaluate the purity of the recmSLLP1 preparation, an aliquot of the purified protein was separated by 1-D electrophoresis and the gel was silver stained and blotted with anti-his antibody. A prominent band of about 15 kDa and a much fainter putative dimmer at approximately 30 kDa were noted. These results indicated that the recmSLLP1 preparation used for this study was highly purified.

The specificity of the antibody generated in guinea pigs against recmSLLP1 was examined by western blotting against both the recombinant immunogen and mouse sperm proteins. The immune sera recognized the 15 kDa recombinant SLLP1 as well as the putative 30 kDa dimmer found in the preparation, while the preimmune serum as well as the serum from guinea pigs injected with adjuvant alone showed no immunoreactivity with recSLLP1. In mouse sperm extracts the immune serum reacted only with an approximately 15 kDa band while the serum from guinea pigs injected with adjuvant alone as well as preimmune sera showed no reactivity. These results indicated that a specific immunoreagent had been generated to the recmSLLP1 that gave a single band on sperm protein extracts and suggested that mSLLP1 dimerization does not occur in sperm although a small amount does occur during E. coli expression. It also indicated that E. coli expressed mSLLP1 after affinity purification and refolding contained sufficient numbers of immunogenic epitopes to generate antibodies cross reactive with the native mSLLP1.

Mouse SLLP1 is Associated with Mouse Sperm Acrosome and Equatorial Segment

Indirect immunofluorescence analysis of fixed mouse spermatozoa using guinea pig IgG against recmSLLP1 localized mSLLP1 mainly to the anterior acrosome of non-capacitated spermatozoa. However, 21.5% of non-capacitated spermatozoa showed an equatorial segment distribution of mSLLP1 and 2.5% possessed no staining. Acrosome-reacted sperm, determined by the lack of fluorescence in the acrosome of PNA lectin, displayed an equatorial segment reactivity with recmSLLP1. However, 19% of acrosome-reacted spermatozoa retained an anterior acrosome staining distribution of mSLLP1 and 5% possessed no staining. Therefore, the disappearance of staining in the anterior acrosome appeared to correspond to the appearance of staining in the equatorial segment once capacitation and acrosome reaction occurred. This may suggest that the equatorial segment SLLP1 is either masked in acrosome-intact sperm or undergoes redistribution from the anterior acrosome to the equatorial segment following the ionophore-induced acrosome reaction. Importantly, confocal analysis showed a clear equatorial segment localization of mSLLP1 in mouse capacitated spermatozoa bound to mouse eggs, emphasizing the concept that this protein may be involved in sperm-oolema binding.

RecmSLLP1 and Anti-recmSLLP1 Serum Inhibit Fertilization of Mouse Cumulus Intact Eggs

To determine the role of mSLLP1 during fertilization, both spermatozoa and cumulus intact oocytes were pre-incubated with anti-recmSLLP1 serum or preimmune serum for 45 min prior to insemination. Fertilization was conducted in the presence of the antibody and six hours later the eggs were relocated in 100 μl drops of fertilization medium and incubated overnight. In the groups treated with the immune sera at 1:10 or 1:50 dilutions, the percentage of two cells embryos was significantly reduced (61% and 17% inhibition respectively). However a significant effect was not observed at a 1:100 dilution (Table 1A).

Cumulus intact oocytes were then incubated with two concentrations of recmSLLP1, which was present during the fertilization process. Treatment of the cumulus-oocyte complexes with 200 μg/ml recmSLLP1 reduced the fertilization rate from 45% in the control group to 12% in the recmSLLP1 treated group, whereas no significant difference was observed on the percentage of fertilization between the control group and the group treated with 50 μg/ml recombinant protein, although a reduction was noted. Moreover, oocytes pre-incubated in the presence of recePAD (200 μg/ml), an egg cytoplasmic protein used as a control recombinant protein, showed no reduction in fertilization (Table 1B). Taken together, these results suggested that mSLLP1 plays a role in fertilization and prompted a dose-ranging study of gamete fusion. For the data presented in Tables 1A and 1B, the sera or the recombinant proteins were present during fertilization. Two cells embryos were scored as fertilized after 24 h. (*) P≦0.05 (Anova test). Both anti-recmSLLP1 sera and the recmSLLP1 significantly inhibit in vitro fertilization in the mouse.

TABLE 1A
Antibodies against SLLP1 Reduce Fertilization

| | # of | | # of | # of non | % |
|---|---|---|---|---|---|
| | experi- | total # | 2 cells | fertilized | Fertili- |
| Treatment | ments | of eggs | embryo | eggs | zation |
| PI 1:100 | 3 | 20 | 15 | 5 | 75 |
| I 1:100 | 3 | 20 | 14 | 6 | 70 |
| PI 1:50 | 3 | 38 | 27 | 11 | 71 |
| I 1:50 | 3 | 46 | 27 | 19 | 59 * |
| PI 1:10 | 6 | 64 | 43 | 21 | 67 |
| I 1:10 | 6 | 80 | 21 | 59 | 26 * |

Table 1A Different concentrations of decomplemented recmSLLP1 preimmune (PI) or immune (I) sera were added to both gametes 45 min prior to insemination.

TABLE 1B
Recombinant SLLP1 Competitor Reduces Fertilization

| | # of | | # of | # of non | % |
|---|---|---|---|---|---|
| | experi- | total # | 2 cells | fertilized | Fertili- |
| Treatment | ments | of eggs | embryo | eggs | zation |
| Control | 3 | 20 | 11 | 9 | 55 |
| recmSLLP1 | 3 | 52 | 25 | 27 | 48 |
| 50 μg/ml | | | | | |
| Control | 3 | 29 | 13 | 16 | 45 |
| recmSLLP1 | 3 | 64 | 8 | 56 | 12 * |
| 200 μg/ml | | | | | |
| recePAD | 3 | 24 | 16 | 8 | 67 |
| 200 μg/ml | | | | | |

Table 1B Different concentrations of recmSLLP1 or PBS were added to the oocytes 45 min prior to insemination with untreated capacitated mouse spermatozoa. In some cases, oocytes were pre-incubated in the presence of 200 μg/ml of a cytoplasmic oocyte protein, recePAD (negative control) for 45 min.

Mouse SLLP1 has a Role in Sperm-Egg Binding

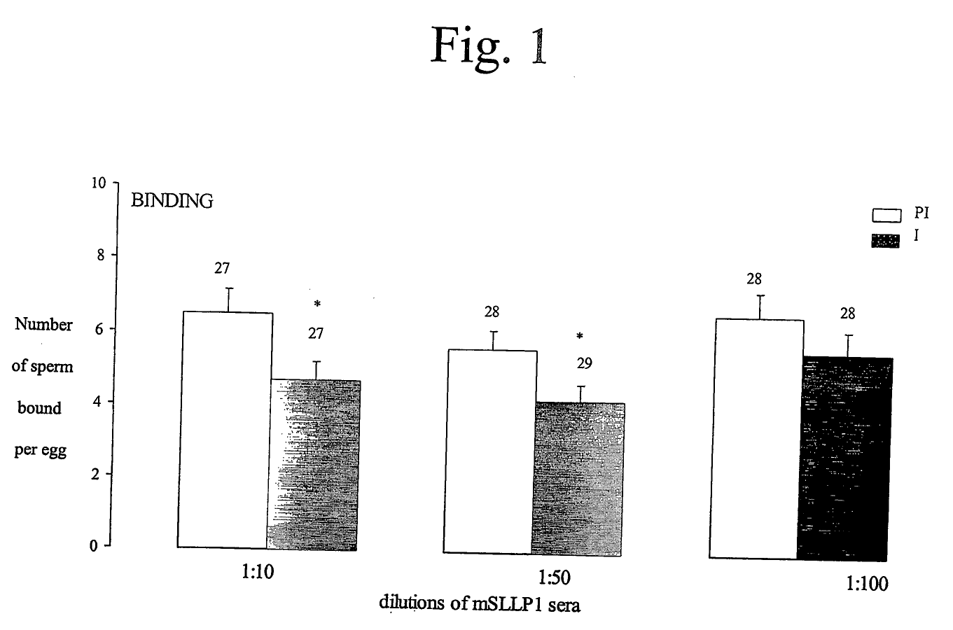

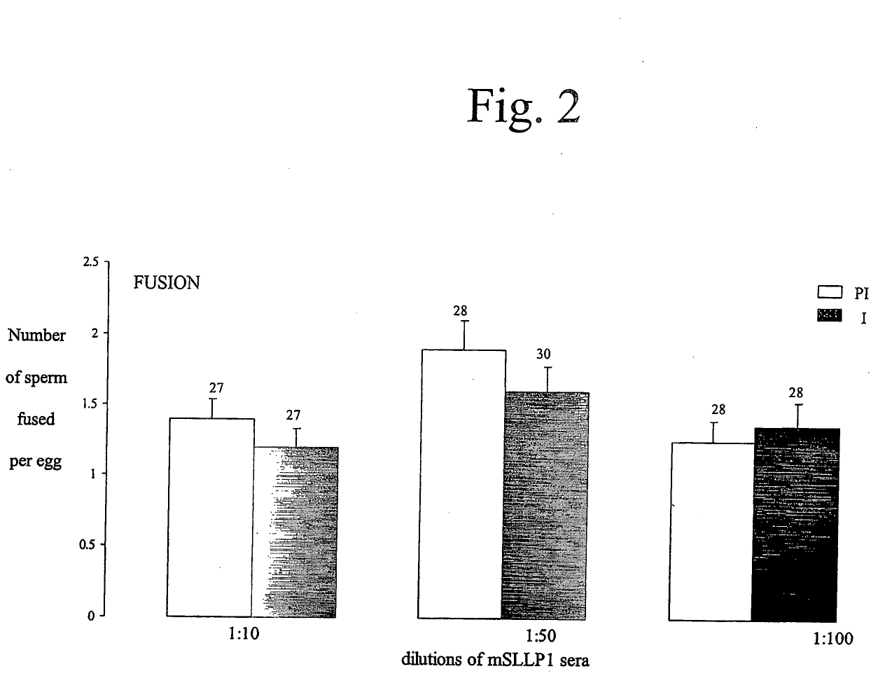

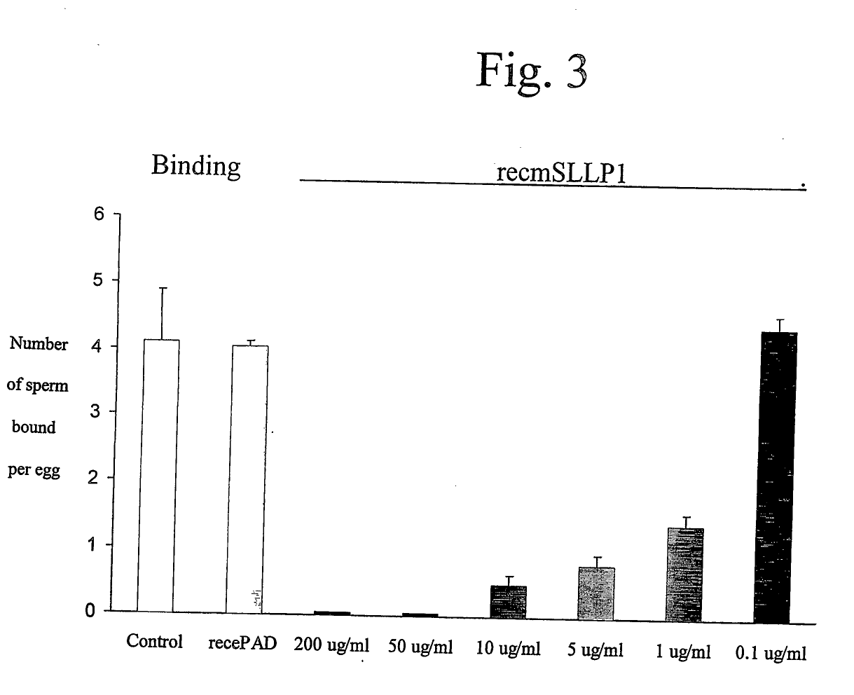

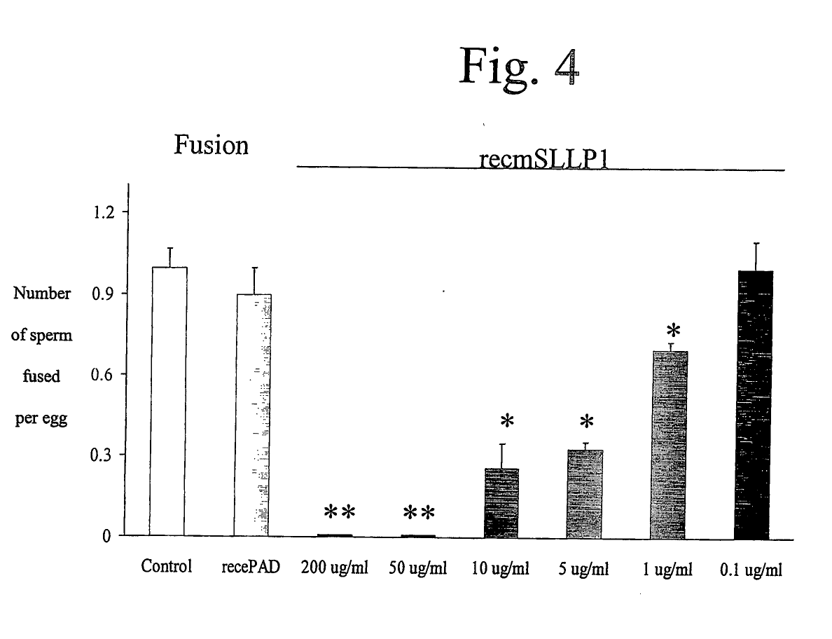

Having noted a significant inhibition of fertilization by recmSLLP1 protein as well as antibodies to recmSLLP1 using cumulus-egg complexes, we were interested in determine the stage in the fertilization cascade at which mSLLP1 exerted its effects. Therefore, we tested whether anti-recmSLLP1 serum and recmSLLP1 protein would block sperm-egg binding or fusion in zona-free mouse eggs by capacitated mouse sperm. Significant inhibition on binding (see FIG. 1) but not fusion (see FIG. 2) was observed when both gametes were co-incubated in the presence of 1:10 and 1:50 dilutions of anti-recmSLLP1 immune sera, whereas 1:100 dilution had no significant effect. The most striking effect was observed when zona-free mouse eggs were incubated with different concentrations of recmSLLP1 (0.1-200 μg/ml) and then inseminated with untreated capacitated mouse spermatozoa. The incubation of oocytes with recmSLLP1 produced a concentration-dependent decrease in the percentage of spermatozoa bound (see FIG. 3) or fused (see FIG. 4) to the egg, with a significant effect observed at 1 μg/ml and 100% inhibition at 50 μg/ml. Zona-free mouse oocytes incubated in the absence of recmSLLP1 or in the presence of recePAD at 200 μg/ml were used as controls. No inhibition was detected when recePAD was tested at 200 μg/ml. No differences were observed in the percentages of motile spermatozoa compared to the controls suggesting that anti-remSLLP1 or recmSLLP1 protein did not affect sperm motility but oolema binding and subsequent fusion. Taken together, these results support the participation of mSLLP1 in the binding event at the mouse egg surface prior to fertilization.

Mouse SLLP1 has Complementary Binding Sites on Unfertilized and Fertilized Oocytes

To study the possible localization of mSLLP1 -binding sites on the egg surface, unfertilized oocytes along with in vitro fertilized oocytes at the pronuclear stage, were incubated with purified recombinant protein mSLLP1 for 45 min, washed and then exposed to anti-recmSLLP1. Unfertilized oocytes exhibited fluorescent labeling within the perivitelline space and over much of the oocyte surface. However, an area devoid of fluorescence was consistently detected. Hoesch staining revealed that this negative area was always associated with the area of the oocyte plasma membrane overlying the metaphase plate. Thus, mSLLP1-binding sites were restricted to the fusogenic region of the egg, which additionally suggested a role for mSLLP1 interaction in sperm-egg binding. In contrast, those oocytes with or without zona pellucida that had been fertilized in vitro, exhibited a patchy and strong fluorescence localized over the entire egg surface. Oocytes subjected to three different treatments were used as controls and none of them presented evidence of fluorescence including oocytes lacking exposure to recmSLLP1, but only exposed to anti-recmSLLP1, oocytes incubated with recmSLLP1 and then exposed to preimmune sera, and oocytes incubated with recePAD and then incubated with the respective specific antibody.

Confocal analysis was performed to refine the localization of mSLLP1 binding sites. Evaluation of unfertilized oocyte cross sections showed that mSLLP1-binding sites localized predominantly to the perivitelline space. A weak fluorescent labeling was also observed on egg's zona pellucida, although at this time we are unable to establish whether this weak fluorescence is due to specific binding. Western blot analysis along with zona binding assays will help to elucidate this issue. In contrast, no fluorescence was detected in the perivitelline space of fertilized eggs but a pattern of patches through out the oolemma was a distinct characteristic.

C Lysozymes do not Block Gamete Fusion Nor Bind to the Mouse Egg

Mouse SLLP1 is a lysozyme-like protein that shares most of the characteristics of c lysozymes including five out of six substrate-binding residues. To evaluate the binding properties of c lysozymes in sperm-egg binding we tested whether human and chicken lysozymes would block binding of zona-free mouse eggs by capacitated mouse sperm. No significant effect on binding or fusion was observed at 50 or 100 μg/ml of native proteins. Accordingly to this functional assay, mouse oocytes did not show a positive fluorescence staining when incubated with human or chicken lysozymes and their respective antibodies.

### DISCUSSION

The results of this study indicate that mSLLP1 is the authentic orthologue of hSLLP1, which shares similar characteristics to the lysozyme family. The mouse SLLP1 orthologue presented here shared 82.8% similarity and 75.8% identity to human SLLP1 in the processed region of the protein, shared several characteristics, including preservation of the critical substrate binding residues for N-acetylglucosamine and preserved the two mutated catalytic residues. Search of the mouse genome database further showed no genes with greater homology to hSLLP1. Together these considerations support the conclusion that the authentic murine orthologue of human

In mammalian fertilization all sperm that have passed through the zona pellucida are acrosome-reacted, and acrosome intact sperm do not fuse with the oolemma (Yanagamachi, R. Adv. Biophys, Vol. 37:49-89, 2003). Fusion between the sperm plasma membrane and the oolemma begins with the plasma membrane domain that overlies the equatorial segment. The molecular mechanisms whereby this specific domain becomes fusogenic are presently unknown, however hypothetical pathways have been proposed (R. Yanagimachi 1994 Frontiers in Endocrinology Vol. 8, p. 15) including the postulate that contents of the acrosomal matrix released during the acrosome reaction redistributes to the equatorial segment. Mouse SLLP1 localizes mainly to the anterior acrosome in acrosome intact mouse sperm and relocates to the equatorial segment following the ionophore-induced acrosome reaction. We do not yet know if this relocation involves redistribution to the equatorial matrix where mSLLP1 is exposed in the cleft between inner and outer acrosomal membranes or redistributes to the overlying plasmalemma or both. Accordingly, spermatozoa that were capacitated and bound to the plasma membrane of the egg still retained mSLLP1 protein, suggesting a role for mSLLP1 in membrane binding.

Using cumulus intact oocytes, fertilization in vitro was blocked when both gametes were incubated with the antisera against recmSLLP1 during the fertilization process. Another important finding from the in vitro fertilization studies was that when recmSLLP1 was present during the fertilization process, fertilization was abolished, suggesting that mSLLP1 is involved in fertilization. Note that as the recmSLLP1 was dialyzed against PBS, PBS was also added to the control oocytes. As a result, the percentage of fertilization obtained for the PBS group was lower than expected (50% vs 75%).

The zona-free sperm-egg binding and fusion assay demonstrated that antibody against recmSLLP1 significantly inhibited binding but not fusion of sperm with zona-free mouse eggs. Further, oocytes incubated in the presence of recmSLLP1 inhibited sperm binding to the oolema in a concentration-dependent manner. Perhaps, the most important observation of this study is that 100% inhibition on sperm-egg binding was observed when both gametes were incubated with 50 μg/ml recmSLLP1. Under our experimental conditions, the effect of recmSLLP1 observed on the fusion process was at the binding but not at the fusion level because spermatozoa that were able to bind where then able to fuse to the oolema. Taken together, the in vitro data suggest that mSLLP1 has a role in fertilization, at least during the sperm-egg binding event.

Although, a large number of potential candidate binding partners for sperm-egg interaction have been described, the precise roles of these molecules have not been firmly established. Therefore, the identification of additional molecules that participate in gamete interactions, particularly those retained after the acrosome reaction, could greatly enhance the understanding of fertilization. SLLP1 is an attractive addition to this group and our findings showed that mSLLP1-binding sites exist on the oolemma. The binding sites were localized to the perivitelline space and over the entire surface of mouse unfertilized oocytes, with the exception of a negative area coincident with the region of plasma membrane overlying the meiotic spindle, region where fusion rarely takes place. This conclusion was supported by the disappearance of fluorescence of the negative region on the fertilized oocytes. Moreover, discrete patches of the fluorescent labeling were observed over the entire egg surface on fertilized oocytes.

### EXAMPLE 4

### Effects of Cross Species SLLP Polypeptide on Mammalian Sperm/Egg Binding

Applicants have demonstrated that the sperm cells of different mammalian species are capable of binding to hamster eggs. More particularly, mouse sperm, in addition to hamster sperm, are capable of binding to hamster eggs during in vitro experiments. Recombinant human SLLP1 has been produced using both yeast and E. coli expression systems and the synthesized protein was used to investigate whether or not it could bind to mammalian eggs. Both recombinantly produced human SLLP1 proteins have been found to bind to zona pellucida free and zona pellucida intact mouse eggs as well as zona pellucida free and zona pellucida intact hamster eggs, using 100 μg/ml of recombinant SLLP1 and the procedures described in Example 3. Furthermore, as a control, applicants have demonstrated that human lysozyme fails to bind to mouse eggs under similar conditions as those used for the SLLP1 binding experiments.

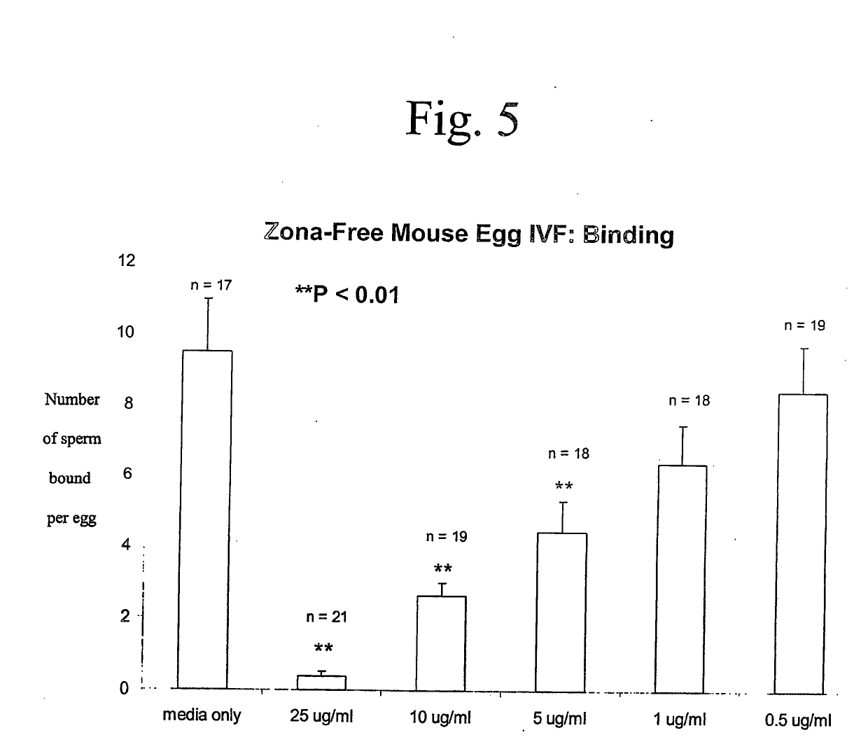

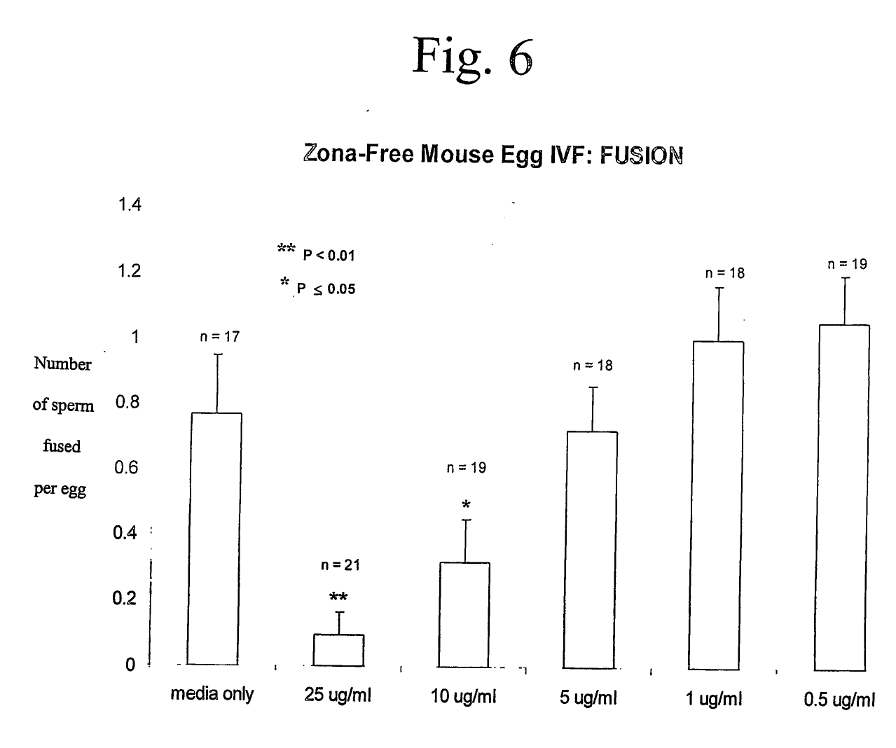

The effect of E. coli expressed recombinant SLLP1 protein on mouse sperm egg binding and fusion was then investigated using the procedures described in Example 3. Briefly, recombinant cells carrying the SLLP1 gene were induced with IPTG. Inclusion bodies were recovered and solubilized in urea or guanidine-HCL. The proteins were then affinity purified on a His binding resin. The resin was washed with imidazole. Complete removal of urea/guanidine from the column was accomplished using a gradient elution in 3 to 6 hours. Elution of folded (soluble) protein was accomplished by imidazole in the absence of urea. The elution media was then exchanged with a buffer suitable to the eluted protein. As shown in FIG. 5 and 6, human recombinant SLLP1 (hrSLLP1) inhibited mouse sperm-egg binding and fusion in a dosage dependent manner. Accordingly, SLLP polypeptides appear capable of interfering with sperm egg binding and fusion in a cross species manner, making these proteins themselves suitable for administration as the active contraceptive agent.

1. A composition for inhibiting binding of sperm to oocytes, said composition comprising a polypeptide, wherein said polypeptide comprises an amino acid sequence selected from the group consisting of SEQ ID NO: 2, SEQ ID NO: 4, SEQ ID NO: 6, SEQ ID NO: 8, SEQ ID NO: 10, SEQ ID NO: 12, and bioactive fragments of such sequences.

2. The composition of claim 1 wherein said polypeptide consists of an amino acid sequence selected from the group consisting of SEQ ID NO: 16, SEQ ID NO: 17, SEQ ID NO: 18, SEQ ID NO: 19, SEQ ID NO: 20 and SEQ ID NO: 21.

3. The composition of claim 1 wherein said polypeptide consists of a peptide fragment of an amino acid sequence selected from the group consisting of SEQ ID NO: 16, SEQ ID NO: 17, SEQ ID NO: 18, SEQ ID NO: 19, SEQ ID NO: 20 and SEQ ID NO: 21:

4. The composition of claim 1 wherein the polypeptide is SEQ ID NO: 2, SEQ ID NO 16, or a bioactive fragment of SEQ ID NO: 2.

5. The composition of claim 4 further comprising a second amino acid sequence selected from the group consisting of SEQ ID NO: 4, SEQ ID NO: 6, SEQ ID NO: 8, SEQ ID NO: 10, SEQ ID NO: 12, and bioactive fragments of such sequences.

6. The composition of any of claims 1-5 further comprising a pharmaceutically acceptable carrier.

7. The composition of any of claims 1-5 wherein said polypeptide is labeled, either directly or indirectly with a detectable label.

8. The composition of any of claims 1-5 further comprising a compound selected from the group consisting of propylene glycol, a monohydric alcohol having 2 to 4 carbon atoms, lactic acid, thioglycol, a fatty acid glyceride, and a sorbitan fatty acid ester.

9. An antibody that specifically binds to a polypeptide selected from the group consisting of SEQ ID NO: 6, SEQ ID NO: 8, SEQ ID NO: 10 and SEQ ID NO: 12.

10. The antibody of claim 9 wherein said antibody is a monoclonal antibody.

11. The antibody of claim 9 or 10 wherein said antibody is labeled, either directly or indirectly with a detectable label.

12. A composition comprising the antibody of claim 11 and a pharmaceutically acceptable carrier.

13. A method for identifying a natural ligand of an SLLP polypeptide, said method comprising the steps of
contacting a human SLLP polypeptide, under physiological conditions, with compounds isolated from oocytes, wherein said SLLP polypeptide comprises an amino acid sequence selected from the group of SEQ ID NO: 6, SEQ ID NO: 8, SEQ ID NO: 10, SEQ ID NO: 12, and bioactive fragments of such sequences;
washing the human SLLP polypeptide to remove unbound and non-specific bound material; and
isolating compounds that remain bound to the human SLLP polypeptide.

14. The method of claim 13 wherein the compounds isolated from oocytes comprise proteins extracted from mammalian oocytes.

15. The method of claim 14 wherein the human SLLP polypeptide is immobilized on a solid surface.

16. A method of decreasing the binding of mammalian sperm cell to a mammalian oocyte, said method comprising the steps of contacting the gametes of a mammalian species with a composition comprising an inhibitor of SLLP activity.

17. The method of claim 16 wherein the administered composition comprises an antibody that specifically binds to a SLLP polypeptide.

18. The method of claim 16 wherein the administered composition comprises an amino acid sequence selected from the group consisting of SEQ ID NO: 2, SEQ ID NO: 4, SEQ ID NO: 6, SEQ ID NO: 8, SEQ ID NO: 10, SEQ ID NO: 12, and bioactive fragments of such sequences.

19. The method of claim 18 wherein the administered composition comprises an amino acid sequence selected from the group consisting of SEQ ID NO: 6, SEQ ID NO: 8, SEQ ID NO: 10, SEQ ID NO: 12, and bioactive fragments of such sequences.

20. The method of claim 19 wherein the administered composition comprises an amino acid sequence selected from the group consisting of SEQ ID NO: 18, SEQ ID NO: 19, SEQ ID NO: 20 and SEQ ID NO: 21.

21. A recombinant human SLLP gene construct, said construct comprising a non-native promoter operably linked to a nucleic acid sequence selected from the group consisting of SEQ ID NO: 5, SEQ ID NO: 7, SEQ ID NO: 9 and SEQ ID NO: 11.

22. A transgenic cell comprising the construct of claim 21.

23. A composition for inducing an immune response in a mammalian species, wherein the immune response decreases the fertility of the individual, said composition comprising an epitope of a SLLP polypeptide selected from the group consisting of SEQ ID NO: 6, SEQ ID NO: 8, SEQ ID NO: 10, SEQ ID NO: 12; and
a pharmaceutically acceptable carrier.

24. The composition of claim 23 further comprising an adjuvant.

25. A method of decreasing the fertility of mammalian species, said method comprising the steps of inducing an immune response in said mammalian species by administering a composition comprising an epitope of a SLLP polypeptide selected from the group consisting of SEQ ID NO: 6, SEQ ID NO: 8, SEQ ID NO: 10 and SEQ ID NO: 12 to said mammalian species.

26. A composition for modulating the fertility of an individual, said composition comprising a peptide fragment of an amino acid sequence selected from the group consisting of SEQ ID NO: 2 and SEQ ID NO: 4.

27. The composition of claim 26 wherein the composition comprises an eight to fifteen amino acid sequence that is identical to an eight to fifteen amino acid sequence of SEQ ID NO: 6, SEQ ID NO: 8, SEQ ID NO: 10 and SEQ ID NO: 12.

28. The composition of claim 26 or 27 wherein the peptide fragment is conjugated to a fatty acid.

29. A method for imaging oocytes in vivo, said method comprising the step c administering to a female a composition comprising an amino acid sequence selected from the group consisting of SEQ ID NO: 2, SEQ ID NO: 4, SEQ ID NO: 6, SEQ ID NO: 8, SEQ ID NO: 10 and SEQ ID NO: 12, wherein said amino acid sequence is labeled, either directly or indirectly with a detectable label.

30. The method of claim 29 wherein the composition is administered vaginally.
